(19) United States
(12) Patent Application Publication
(10) Pub. No.: US 2024/0248078 A1
(43) Pub. Date: Jul. 25, 2024

(54) Compositions For and Methods of Evaluating Gap Junction Formation and Function

(71) Applicant: Duke University, Durham, NC (US)

(72) Inventors: Kafui Dzirasa, Durham, NC (US); Elizabeth Ransey, Durham, NC (US)

(21) Appl. No.: 18/564,491

(22) PCT Filed: Jun. 3, 2022
(86) PCT No.: PCT/US22/32087

§ 371 Date: Nov. 27, 2023

Related U.S. Application Data
(60) Provisional application No. 63/197,010, filed on Jun. 4, 2021.

(51) Int. Cl. G01N 33/50

(52) U.S. Cl. CPC G01N 33/5076; G01N 33/5032; G01N 2333/976

(57) ABSTRACT

Disclosed herein are compositions for use in methods of evaluating gap junction formation, methods of interrogating the docking interactions between connexins, and methods of high-throughput quantification of gap junction hemichannel docking.

### CROSS-REFERENCE TO RELATED APPLICATION

This application claims priority to U.S. Provisional Application No. 63/197,010 filed 4 Jun. 2021, which is incorporated herein in its entirety.

### STATEMENT REGARDING FEDERALLY SPONSORED RESEARCH OR DEVELOPMENT

This invention was made with government support under R21 EY029451-01 awarded by the National Eye Institute. The government has certain rights in the invention.

### REFERENCE TO THE SEQUENCE LISTING

The Sequence Listing submitted 3 Jun. 2022 as a text file named “22_2045_WO_Sequence_Listing_ST25”, created on 3 Jun. 2022 and having a size of 684 kilobytes is hereby incorporated by reference pursuant to 37 C.F.R. § 1.52(e)(5).

### BACKGROUND

Gap junctions are membrane spanning channels that connect the cytoplasm of apposed cells, allowing for the passage of small molecules and ions. Gap junctions (GJs) play critical roles in tissue homeostasis, cellular signaling, and intercellular communication (i.e., ionic and small molecule exchange) (Alexander D B, et al. (2003) Curr Med Chem. 10:2045-2058). GJs are composed of connexin (Cx) proteins, which oligomerize into hexameric hemichannels (Falk M M, et al. (1997) EMBO J. 16:2703-2716; AhmadS, et al. (1999) Biochem J. 339(Pt 2):247-253) and dock with compatible hemichannels on adjacent cells to form intercellular pores (Perkins G A, et al. (1998) J Mol Biol. 277:171-177). Connexins play significant roles in the normal physiology of the tissues in which they are expressed, thus, mutations in any of the Cx genes, can lead to pathological changes—including congenital sensorineural deafness (Lee J R, et al. (2009) Expert Rev Mol Med. 11:e35; Scott C A, et al. (2011) Biochem J. 438:245-254); Charcot-Marie-Tooth disorder (Nelis E, et al. (1999) Human Mutation. 13:11-28), and Oculodentodigital dysplasia (ODDD) (Laird D W. (2014) Febs Letters. 588:1339-1348). Additionally, expression level changes have been associated with epileptic conditions (Jin M M, et al. (2011) Neurosci Bull. 27:389-406; Naus C C, et al. (1991) Exp Neurol 0.111:198-203) and several cancers (Mesnil M. (2002) Biol Cell. 94:493-500).

Gap junctions are comprised of connexin proteins (e.g., currently 21 isoforms) that assemble into hexameric hemichannels on each cell and selectively dock to hemichannels on adjacent cells.

The 21-isoform family of human Cxs (Sohl G, et al. (2003) Cell Commun Adhes. 10:173-180; Sohl G, et al. (2004) Cardiovasc Res. 62:228-232) are expressed in virtually every tissue in the body (Saez J C, et al. (2003) Physiol Rev. 83:1359-1400; Goodenough D A, et al. (1996) Annu Rev Biochem. 65:475-50) with distinct but often overlapping distributions (Laird D W. (2006) Biochem J. 394:527-543). Since there is some degree of docking and oligomerization compatibility amongst different isoforms, the diversity of composition and functional features of the resultant GJs is substantial. However, major gaps exist in knowledge of how different isoforms interact with each other within and across different tissues and cell types.

Thus, there remains an urgent need for direct, high-throughput, accurate, and fast analyses of gap junction formation and function. Consequently, the present disclosure provides compositions for and methods of evaluating gap junction formation, interrogating the docking interactions between connexins, and high-throughput quantification of gap junction hemichannel docking.

### BRIEF DESCRIPTION OF THE FIGURES

FIG. 1A-FIG. 1B shows Flow Enabled Tracking of Connexosomes in HEK cells. (FETCH). FIG. 1A is a schematic of connexin protein life cycle. Connexin proteins assemble into hexameric hemichannels that localize to the plasma membrane of individual cells. Hemichannels diffuse laterally and dock to hemichannels on the plasma membrane of an apposed cell, forming a functional gap junction. Gap junctions concentrate at gap junction plaques which may be comprised of tens to tens of thousands of gap junctions. Cellular down-regulation of gap junction intercellular communication results in the internalization of central portions of gap junction plaques as double-bi-layer vesicular structures called connexosomes. Connexosomes are comprised of fully-docked gap junctions, thus differential fluorescent labeling of counterpart hemichannels results in dual-labeled connexosomes. Here, the lighter and darker gray tags indicate different colors of fluorescent tags. FIG. 1B is a schematic representing expected results of flow cytometry analysis for different combinations of connexin proteins. Paired connexin hemichannels that successfully dock are expected to elicit a strong flow cytometry phenotype representative of two-color connexosomes generation (double positive cells). Paired connexin hemichannels that fail to dock, do not generate connexosomes and, thus, do not generate a corresponding flow cytometry indicative of double positive cells. Here, hemichannel docking is indicated by the observation of two-color/dual-labeled annual gap junctions.

FIG. 2A-FIG. 2E shows fluorescence exchange phenotype is mediated by connexin proteins. FIG. 2A is a schematic of FETCH experimental set-up involving the co-plating and incubation of individually transfected cell populations. FIG. 2B shows confocal images of co-plated cytoplasmic-fluor and Cx43-fluor expressing HEK 293FT cells. FIG. 2C shows flow cytometry profiles of co-plated cytoplasmic-FP and Cx43-FP cells. Fluorescence exchange can be observed as the cells that display both fluorescent proteins, despite only expressing one construct (Q2). FIG. 2D shows cells sorted from either side of the double-positive quadrant of the co-plated Cx43-mEmerald/Cx43-mApple sample (i.e., higher expressing in mEmerald, higher expressing in mApple). Insets show internalized vesicular structures of double-positive sorted cells. Dual-labeled structures are consistent with connexosomes.

FIG. 3A-FIG. 3C shows that FETCH can be used to evaluate homotypic and heterotypic docking of multiple connexin isoforms. FIG. 3A shows FETCH score distributions of known positive and known negative connexin protein combinations. The FETCH score is defined as the proportion of Q2, double-positive cells over all fluorescent cells (Q2/Q1+Q2+Q3). FIG. 3B shows homotypic FETCH scores of all human connexin isoforms. FIG. 3C shows FETCH scores of all paired combinations for Cx26, Cx32, Cx36, Cx43, and Cx45.

FIG. 4A-FIG. 4E show that FETCH can be used to evaluate docking ability of connexin mutant proteins. FIG. 4A is a schematic representation (left side) and FETCH scores (right side) of 10 Oculodentodigital dysplasia (ODDD)-associated Cx43 mutations (in black text) and 5 random alanine mutations (in gray text). FIG. 4B shows that mutation colors reflect docking (green dots) and non-docking (red dots) as determined by statistical analysis of FETCH scores. FIG. 4C is a schematic of GAP-FRAP (fluorescence recovery after photobleaching). Calcein-AM cell permeant dye is added to confluent cells transiently transfected with Cx43-WT and Cx43-mutant constructs. Upon permeating cells, calcein-AM is cleaved by intracellular esterases, making it cell-impermeant; residual dye is removed via repetitive washes. Individual cells containing calcein are bleached using a 488 nm laser, and the recovery of cellular fluorescence over time, mediated by gap junctions, is recorded. FIG. 4D shows the GAP-FRAP analysis of ODDD-associated and random Cx43 mutant proteins. Bar graph depicts maximum fluorescence recovery reached at the end of the recovery time interval (4 mins). Inset shows full initial, bleach and recovery traces for each protein, in addition to corresponding 95% confidence intervals. Mutation colors are carried over from the associated FETCH analysis for each mutation as docking (green bars and traces) and non-docking (red bars and traces); Cx43-WT and Cx43-WT+Inhibitor (100 μM carbenoxolone) are depicted as black. FIG. 4E shows that FETCH scores correlate with FRAP-determined functionality.

FIG. 5A-FIG. 5B shows that FETCH can evaluate connexin mutations that confer new docking features. FIG. 5A is a schematic of a chimeric connexin construct consisting of Cx43 with the extracellular loops (ELs) of Cx26. FIG. 5B are the flow cytometry plots demonstrating that the Cx43-ELs-Cx26 construct docks with Cx26, but not Cx43.

FIG. 6 shows summary of FETCH method contribution to the evaluation of connexin protein interactions. Using FETCH docking analysis coupled with a functional assay such as dye transfer can help distinguish incompatible hemichannels and non-functional, docked channels.

FIG. 7A-FIG. 7B show the expression of Cx43-mEmerald in different cell lines. FIG. 7A shows confocal images of HEK 293FT, HEK 293T, HEK 293, and HeLa cell lines transiently transfected with Cx43-mEmerald with the nuclei stained with DAPI. FIG. 7B shows a western blot of different cell lines transiently transfected with Cx43-mEmerald.

FIG. 8A-FIG. 8B shows the FETCH analysis pipeline and pilot data for FETCH score distributions. FIG. 8A provides automated gating pipeline that uses sequential kde- and std-based gating approaches to obtain a FETCH score from a single raw .fcs file. FIG. 8B shows FETCH score distributions of known positive and negative combinations used for statistical evaluation. The 95% confidence interval for the known positive and known negative distributions are shown in green and red, respectively.

### BRIEF SUMMARY

Disclosed herein are connexin proteins. Disclosed herein is a fluorescently-labeled connexin protein. Disclosed herein is a fluorescently-labeled connexin construct.

Disclosed herein is an isolated nucleic acid molecule comprising a sequence that encodes the sequence set forth in any one of SEQ ID NO:01-SEQ ID NO:21, or a fragment thereof, or a mutant thereof.

Disclosed herein is an isolated nucleic acid molecule comprising a sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof, or a mutant thereof. Disclosed herein is an isolated nucleic acid molecule comprising a sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof, or a mutant thereof. Disclosed herein is an isolated nucleic acid molecule comprising a sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, or a mutant thereof.

Disclosed herein is an isolated nucleic acid molecule comprising the sequence set forth in any one of SEQ ID NO:22 to SEQ ID NO:42, or a fragment thereof, or a mutant thereof. Disclosed herein is an isolated nucleic acid molecule comprising any sequence set forth in Table 6. Disclosed herein is an isolated nucleic acid molecule comprising a sequence that encodes the sequence set forth in any one of SEQ ID NO:01-SEQ ID NO:21, or a fragment thereof, or a mutant thereof, and a sequence encoding a tag and/or label.

Disclosed herein is an isolated nucleic acid molecule comprising a sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof, or a mutant thereof, and a sequence encoding a tag and/or label. Disclosed herein is an isolated nucleic acid molecule comprising a sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof, or a mutant thereof, and a sequence encoding a tag and/or label. Disclosed herein is an isolated nucleic acid molecule comprising a sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, or a mutant thereof, and a sequence encoding a tag and/or label.

Disclosed herein is an isolated nucleic acid molecule comprising the sequence set forth in any one of SEQ ID NO:22 to SEQ ID NO:42, or a fragment thereof, or a mutant thereof, and a sequence encoding a tag and/or label. Disclosed herein is an isolated nucleic acid molecule comprising a sequence that encodes the sequence set forth in any one of SEQ ID NO:01-SEQ ID NO:21, or a fragment thereof, or a mutant thereof, and a sequence encoding a tag and/or label.

Disclosed herein is an isolated nucleic acid molecule comprising the sequence set forth in any one of SEQ ID NO:22-SEQ ID NO:42, or a fragment thereof, or a mutant thereof, and a sequence encoding a tag and/or label. Disclosed herein are cells comprising a disclosed isolated nucleic acid molecule or a disclosed plasmid. Disclosed herein are cells transfected by one or more disclosed nucleic acid molecules. Disclosed herein are cells transfected by an isolated nucleic acid molecule comprising a nucleic acid sequence encoding the sequence set forth in any one of SEQ ID NO:01-SEQ ID NO:21, or a fragment thereof, or a mutant thereof.

Disclosed herein are cells transfected by an isolated nucleic acid molecule comprising a nucleic acid sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof, or a mutant thereof. Disclosed herein are cells transfected by an isolated nucleic acid molecule comprising the nucleic acid sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof, or a mutant thereof. Disclosed herein are cells transfected by an isolated nucleic acid molecule comprising a nucleic acid sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, or a mutant thereof

Disclosed herein are cells transfected by an isolated nucleic acid molecule comprising the sequence set forth in any one of SEQ ID NO:22 to SEQ ID NO:42, or a fragment thereof, or a mutant thereof

Disclosed herein is a kit comprising one or more disclosed isolated nucleic acid molecules, one or more disclosed proteins, one or more disclosed cells, or any combination thereof. Disclosed herein is a kit comprising a disclosed connexin protein. Disclosed herein is a kit comprising a disclosed connexin protein comprising a label and/or a tag. Disclosed herein is a kit comprising one or more disclosed cells. Disclosed herein is a kit comprising one or more disclosed compositions and/or components and/or agents that can be used in any disclosed method.

Disclosed herein is a method of evaluating gap junction formation, the method comprising performing flow enabled tracking of connexosomes in HEK 293FT cells (FETCH). Disclosed herein is a method of interrogating the docking interactions between connexins, the method comprising performing flow enabled tracking of connexosomes in HEK 293FT cells (FETCH).

Disclosed herein is a method of high throughput quantification of gap junction hemichannel docking, the method comprising performing flow enabled tracking of connexosomes in HEK 293FT cells (FETCH).

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Connexins

Gap junctions are multimeric, transmembrane channels that play crucial roles in tissue homeostasis, cellular signaling, and the propagation of electrical current by enabling direct intercellular communication between adjacent, apposed cells via ionic and small molecule exchange (Alexander D B, et al. (2003) Curr Med Chem. 10:2045-2058)). Gap junctions are composed of connexin (Cx) proteins, which oligomerize into hexameric hemichannels (Falk M M, et al. (1997) EMBO J. 16:2703-2716; Ahmad S, et al. (1999) Biochem J. 339(Pt 2):247-253) and dock with compatible hemichannels of adjacent cells at the plasma membrane to form an intercellular pore connecting the two cells (Perkins G A, et al. (1998) J Mol Biol. 277:171-177). As shown in Table 1, connexins comprise a family of integral membrane proteins consisting of 21 unique isoforms in humans (Sohl G, et al. (2003) Cell Commun Adhes. 10:173-180; Sohl G, et al. (2004) Cardiovasc Res. 62:228-232), with at least one isoform expressed in virtually every tissue and major organ in the body (Goodenough D A, et al. (1996) Annu Rev Biochem. 65:475-502) resulting in distinct and overlapping isoform-specific expression patterns. Routinely, single cell-types simultaneously express two or more connexin isoforms, creating both opportunities for functional redundancy and complex, highly-regulated tissue communication (Laird D W. (2006) Biochem J. 394:527-543).

Connexins play significant roles in the normal physiology of the tissues in which they are expressed. Thus, mutations in any of the Cx genes can lead to pathological changes. For example, at least 100 mutations of the Connexin26 (Cx26) encoding gene (GJB2) accounts for half of all worldwide cases of congenital sensorineural deafness (Lee J R, et al. (2009) Expert Rev Mol Med. 11:e35; Scott C A, et al. (2011) Biochem J. 438:245-254). Charcot-Marie-Tooth, a motor and sensory neurodegenerative disorder, is associated with mutations of the Connexin32 (Cx32) gene (GJB1) (Nelis E, et al. (1999) Human Mutation. 13:11-28). At least 73 different mutations of the most ubiquitously expressed connexin, Connexin43 (Cx43) gene (GJA1), are associated with Oculodentodigital dysplasia (ODDD) (Laird D W. (2014) Febs Letters. 588:1339-1348), which is a rare, developmental disorder resulting in numerous morphological anomalies and neurological symptoms (De Bock M, et al. (2013) Front Pharmacol. 4:120). Additionally, aberrant expression levels or regulation of Cx43, the primary connexin expressed in the heart, are associated with cardiac arrythmias in the context of myocardial ischemia (Lerner D L, et al. (2000) Circulation. 101:547-552).

Connexin proteins share a conserved topology consisting of intracellular amino- and carboxy-termini, a cytoplasmic loop, 4 transmembrane (TM) helices, and 2 extracellular loops (ELs). Post-translational modification (e.g., phosphorylation) of the Cx C-terminus regulates transport to and from the plasma membrane (Lampe P D, et al. (2000) Arch Biochem Biophys. 384:205-215) and the N-terminus contributes to channel gating (Lee H J, et al. (2020) Science advances. 6:eaba4996). Motifs in and near the TM domains affect connexin oligomerization and gap junction or hemichannel conductance (Hu X, et al. (2006) Biophysical journal. 90:140-150). The ELs primarily function to impart hemichannel docking specificity (Koval M, et al. (2014) Febs Letters. 588:1193-1204), though EL1 can also contribute to channel properties including permeability and conductance (Oh S, et al. (1999) The J Gen Physiol. 114(3):339-364; Trexler E B, et al. (2000) Biophys J. 79:3036-3051; Bai D L, et al. (2014) Biochem J. 458:1-10). Thus, the primary differentiating features of connexins are trafficking and assembly, oligomerization specificity, docking specificity and permeability or conductance.

The presence of multiple unique Cx isoforms throughout the body diversifies gap junction intercellular communication. For example, all Cxs form homotypic gap junctions (two hemichannels of one isoform (e.g., Cx36/Cx36 gap junctions) between two neurons (Deans M R, et al. (2002) Neuron. 36:703-712). But most isoforms can also form heterotypic channels; that it, two hemichannels of different isoforms (e.g., Cx46/Cx50 gap junctions in the lens (Lampe P D, et al. (2000) Arch Biochem Biophys. 384:205-215)) with compatibility being dictated by motifs in the ELs. Frequently, because each isoform has slightly variable permeability and conductivity, heterotypic channels exhibit a preferred ionic directional flow (rectification). Additionally, within cells in which more than one Cx is expressed, multiple isoforms may be mixed into single hemichannels, called hetero-oligomerized or heteromeric hemichannels (e.g., cardiac Cx43/Cx45 hemichannels (Martinez A D, et al. (2002) Circ Res. 90:1100-1107), with compatibility dictated by motifs within and adjacent to TM regions (Smith T D, et al. (2012) J Membr Biol. 245(5-6):221-230; Koval M, et al. (2014) Febs Letters. 588:1193-1204).

Another important feature of gap junction intercellular communication centers on the lifetime of connexin proteins at the plasma membrane being relatively short with half-lives of ˜1-5 hrs (Laird D W, et al. (1991) Biochem J. 273(Pt 1):67-72; Fallon R F, et al. (1981) J Cell Biol. 90:521-526; Laing J G, et al. (1995) J Biol Chem. 270:26399-26403; Beardslee M A, et al. (1998) Circ Res. 83:629-635). Given the critical utility of Cxs throughout the body, this short half-life indicates that Cxs are maintained in a constant flux of biosynthesis and degradation, which allows a cell to rapidly up-regulate or down-regulate gap junction intercellular communication in response to physiological needs. Importantly, once hemichannels dock, they are virtually inseparable under physiological conditions (Ghoshroy S, et al. (1995) J Membr Biol. 146(1):15-28; Goodenough D A, et al. (1974) J Cell Biol. 61:575-590). Thus, down-regulation of gap junction intercellular communication is achieved by functional gap junction internalization. Specifically, gap junctions are turned over from the plasma membrane via a unique clathrin/dynamin-dependent internalization process (Piehl M, et al. (2007) Mol Biol Cell. 18:337-347; Gumpert A M, et al. (2008) FEBS Lett. 582:2887-2892; Nickel B M, et al. (2008) Biochem Biophys Res Commun. 374:679-682) that results in the internalization of portions of or entire gap junction plaques in the form of double-bilayer vesicular structures, termed annular gap junctions or, more recently, connexosomes (Larsen W J, et al. (1979) J Cell Biol. 83(3):576-587; Mazet F, et al. (1985) Circ Res. 56:195-204; Jordan K, et al. (2001) J Cell Sci. 114:763-773). Connexosomes contain fully-docked gap junctions that are either degraded (Piehl M, et al. (2007) Mol Biol Cell. 18:337-347) or recycled and transported back to the plasma membrane (Boassa D, et al. (2010) Traffic. 11:1471-1486; Vanderpuye O A, et al. (2016) Cell Biol Int. 40:387-396; Bell C L, et al. (2019a) Int J Mol Sci. 20(1):44).

TABLE 1
Genetic Disorders Caused by Human Connexin Mutations.

| Gene | Chrom. | Protein | Disorder or Disorders | OMIM |
|---|---|---|---|---|
| GJA1 | 6q22.31 | Cx43 | Craniometaphyseal dysplasia, autosomal recessive | 218400 |
| | | | Erythrokeratodermia variabilis et progressive | 133200 |
| | | | Oculodentodigital dysplasia | 164200 |
| | | | Oculodentodigital dysplasia, autosomal recessive | 257850 |
| | | | Palmoplantar keratoderma with congenital alopecia | 104100 |
| | | | Syndactyly, type III | 186100 |
| GJA3 | 13q12.11 | Cx46 | Cataract | 601885 |
| GJA4 | 1p34.3 | Cx37 | | |
| GJA5 | 1q21.2 | Cx40 | Atrial fibrillation, familial, 11 | 614049 |
| | | | Atrial standstill, digenic (GJA5/SCN5A) | 108770 |
| GJA8 | 1q21.2 | Cx50 | Cataract | 116200 |
| GJA9 | 1p34.3 | Cx59 | | |
| GJA10 | 6q15 | Cx62 | | |
| GJB1 | Xq13.1 | Cx32 | Charcot-Marie-Tooth neuropathy, X-linked 1 | 302800 |
| GJB2 | 13q12.11 | Cx26 | Bart-Pumphrey syndrome | 149200 |
| | | | Deafness, autosomal dominant 3A | 601544 |
| | | | Deafness, autosomal recessive 1A | 220290 |
| | | | Hystrix-like ichthyosis with deafness | 602540 |
| | | | Keratitis-ichthyosis-deafness syndrome | 148210 |
| | | | Keratoderma, palmoplantar, with deafness | 148350 |
| | | | Vohwinkel syndrome | 124500 |
| | | | Porokeratotic eccrine ostial and dermal duct nevus | |
| GJB3 | 1p34.3 | Cx31 | Deafness, autosomal dominant 2B | 612644 |
| | | | Deafness, digenic, (GJB2/GJB3) | 220290 |
| | | | Erythrokeratodermia variabilis et progressiva | 133200 |
| GJB4 | 1p34.3 | Cx30.3 | Erythrokeratodermia variabilis et progressiva | 133200 |
| GJB5 | 1p34.3 | Cx31.1 | | |
| GJB6 | 13q12.11 | Cx30 | Deafness, autosomal dominant 3B | 612643 |
| | | | Deafness, autosomal recessive 1B | 612645 |
| | | | Deafness, digenic (GJB2/GJB6) | 220290 |
| | | | Ectodermal dysplasia 2, Clouston type | 129500 |
| GJB7 | 6q14.3-q15 | Cx25 | | |
| GJC1 | 17q21.31 | Cx45 | | |
| GJC2 | 1q42.13 | Cx47 | Leukodystrophy, hypomyelinating, 2 | 608804 |
| | | | Spastic paraplegia 44, autosomal recessive | 613206 |
| | | | Lymphedema, hereditary, IC | 613480 |
| GJC3 | 7q22.1 | Cx30.2 | | |
| GJD2 | 15q14 | Cx36 | | |
| GJD3 | 17q21.2 | Cx31.9 | | |
| GJD4 | 10p11.21 | Cx40.1 | | |
| GJE1 | 6q24.1 | Cx23 | | |

Despite the numerous and broad implications of connexin isoforms in human physiology and disease (see Table 1 above), available tools for discerning key attributes such as hemichannel docking and permeability in vitro are currently limited. The most readily available methods for detecting activity (i) often rely on dye transfer (e.g., GAP-FRAP, scrape loading, microinjection, etc.), which in turn depend on the permeability of channels to small molecule dyes such as calcein, carboxyfluorescein, Lucifer yellow, and ethidium bromide, and (ii) use the transfer of these dyes between adjacent cells as indicators of functional gap junctions (Abbaci M, et al. (2008) Biotechniques. 45:33-62). However, very few dyes have been established as gap junction permeant and such dyes are generally connexin isoform, isoform species, and/or pH specific (Elfgang C, et al. (1995) J Cell Biol. 129:805-81). The highest precision and therefore “gold-standard” method for evaluating connexin activity is whole-cell, dual patch clamp measurement (Veenstra R D, et al. (1986) Science. 233:972-974; White T, et al. (1995) Mol Biol Cell. 6(4):459-470), which can quantitatively characterize the electrical coupling (conductance) between two cells mediated by gap junctions with high sensitivity. But this is a very low-throughput, labor intensive, and expensive technique. Thus, what it desperately needed is a higher throughput, universal method for detection/determination of connexin interactions would contribute more comprehensive understanding of connexin biology.

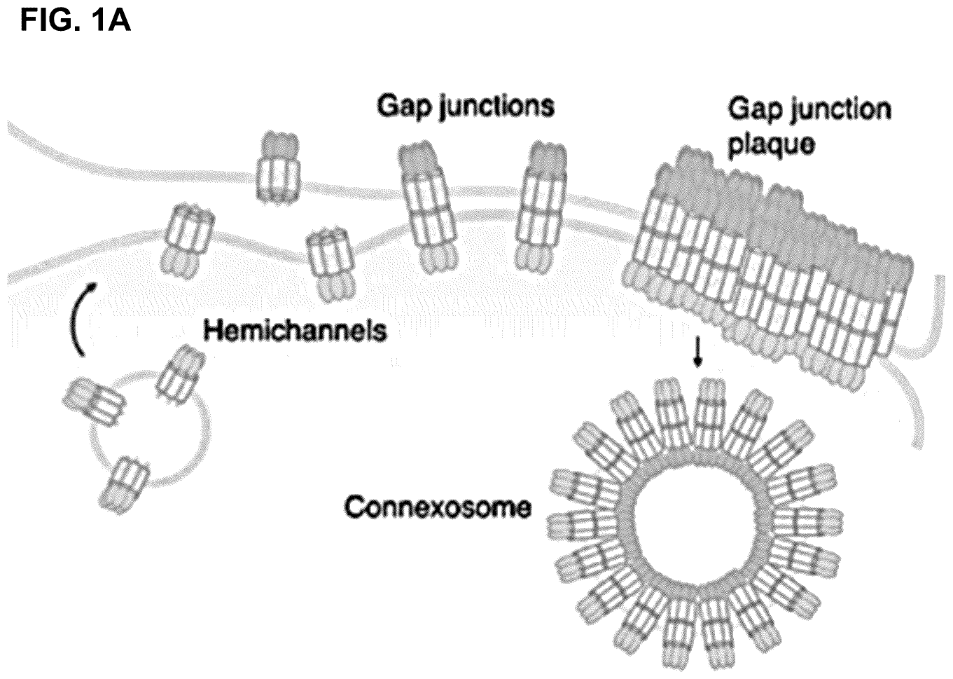

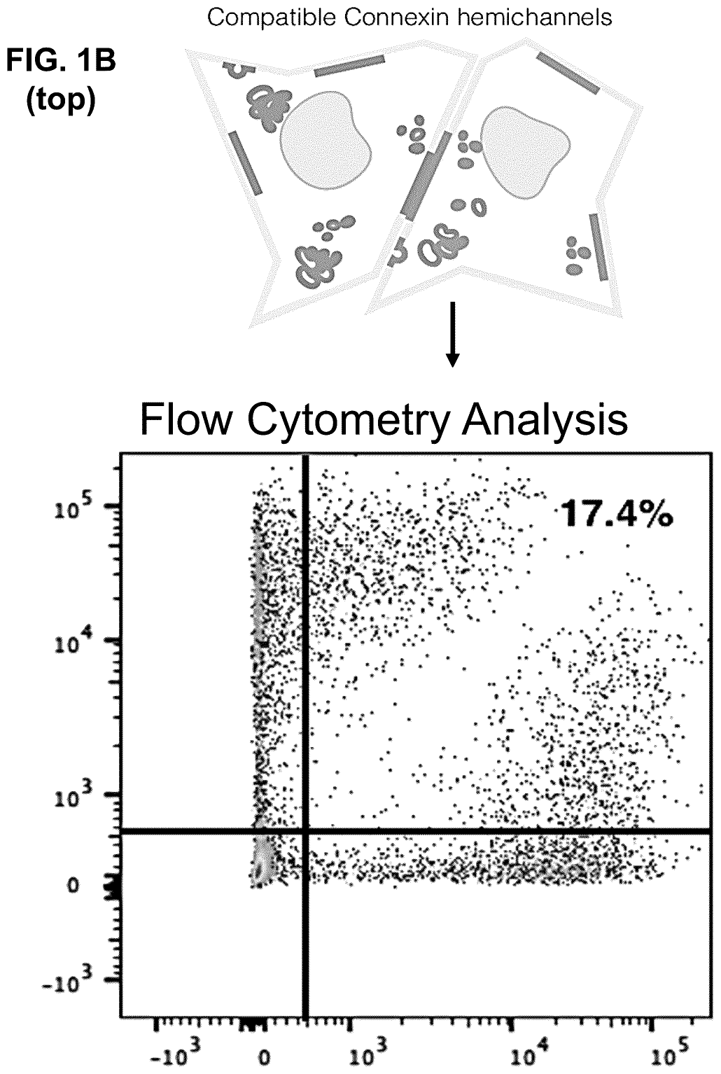

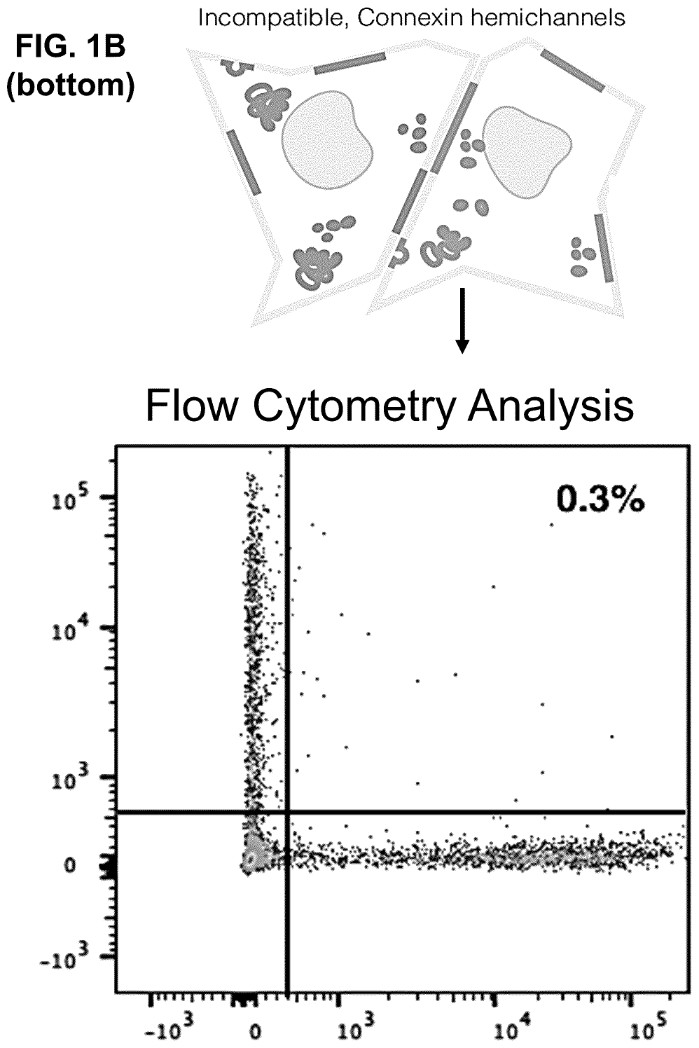

Here, a fluorescence-based, flow cytometry method for the evaluation of gap junction formation was developed. This method, which is dubbed Flow Cytometry Enabled Tracking of Connexosomes in HEK cells (FETCH), is easy, accessible, fast, and scalable for high-throughput analysis. Specifically, FETCH exploits the generation of fluorescent connexosomes between transfected HEK cell populations expressing distinct connexins. As connexosomes are comprised of docked hemichannels from two different cells, the docking capability of different connexin isoforms or mutant proteins can be evaluated by assessing the presence or absence of formed connexosomes, which is a phenotype that is discernable by flow cytometry (FIG. 1A-FIG. 1B).

### B. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered. In an aspect, a subject can be a human subject. In an aspect, a subject can have a disease or disorder characterized by gap junction dysfunction or misfunction. In an aspect, a subject can be suspected of having a disease or disorder characterized by gap junction dysfunction or misfunction.

As used herein, the term “diagnosed” means having been subjected to an examination by a person of skill, for example, a physician, and found to have a condition that can be diagnosed or treated by one or more of the disclosed compositions or by one or more of the disclosed methods. For example, “diagnosed with a disease or disorder characterized by gap junction dysfunction and/or malfunction” means having been subjected to an examination by a person of skill, for example, a physician, and found to have a condition that can be treated by one or more of the disclosed compositions or by one or more of the disclosed methods. For example, “suspected of having a disease or disorder characterized by gap junction dysfunction and/or malfunction” can mean having been subjected to an examination by a person of skill, for example, a physician, and found to have a condition that can likely be treated by one or more of the disclosed compositions or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

A “patient” refers can refer to a subject afflicted with a disease or disorder (e.g., gap junction dysfunction and/or malfunction). In an aspect, a patient can be in the military or can be a veteran. In an aspect, a patient can refer to a subject that has been diagnosed with or is suspected of having a disease or disorder characterized by gap junction dysfunction and/or malfunction. In an aspect, a patient can refer to a subject that has been diagnosed with or is suspected of having a disease or disorder and is seeking treatment or receiving treatment for a disease or disorder (such as a disease or disorder characterized by gap junction dysfunction and/or malfunction). In an aspect, a “patient” can refer to a subject afflicted with a disease or disorder characterized by gap junction dysfunction and/or malfunction. In an aspect, a patient can refer to a subject that has been diagnosed with or is suspected of having a disease or disorder a disease or disorder characterized by gap junction dysfunction and/or malfunction. In an aspect, a patient can refer to a subject that has been diagnosed with or is suspected of having a disease or disorder and is seeking treatment or receiving treatment for a disease or disorder (such as a disease or disorder characterized by gap junction dysfunction and/or malfunction).

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, the phrase “identified to be in need of treatment,” or the like, refers to selection of a subject based upon need for treatment of a disease or disorder characterized by gap junction dysfunction and/or malfunction. For example, a subject can be identified as having a need for treatment based upon an earlier diagnosis by a person of skill and thereafter subjected to treatment for a disease or disorder characterized by gap junction dysfunction and/or malfunction. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of symptoms, complications, issues due to a subject's gap junction dysfunction and/or malfunction. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder characterized by gap junction dysfunction and/or malfunction) or to the level prior to the onset of a disease or disorder characterized by gap junction dysfunction and/or malfunction. Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the onset of gap junction dysfunction and/or malfunction. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the onset of gap junction dysfunction and/or malfunction or a disease or disorder characterized by gap junction dysfunction and/or malfunction. In an aspect, the inhibition or reduction can be 0-25%, 25-50%, 50-75%, or 75-100% as compared to native or control levels or to the subject's level prior to the onset to the onset of gap junction dysfunction and/or malfunction.

The words “treat” or “treating” or “treatment” include palliative treatment, that is, treatment designed for the relief of symptoms rather than the curing of a disease or disorder characterized by gap junction dysfunction and/or malfunction; preventative treatment, that is, treatment directed to minimizing or partially or completely inhibiting the development of a disease or disorder characterized by gap junction dysfunction and/or malfunction; and supportive treatment, that is, treatment employed to supplement another specific therapy directed toward the improvement of a disease or disorder characterized by gap junction dysfunction and/or malfunction. In an aspect, the terms cover any treatment of a subject, including a mammal (e.g., a human), and includes: (i) preventing the undesired physiological change and/or pathological condition from occurring in a subject that can be predisposed to a disease or disorder characterized by gap junction dysfunction and/or malfunction but has not yet been diagnosed as having it; (ii) inhibiting the physiological change and/or pathological condition (a disease or disorder characterized by gap junction dysfunction and/or malfunction); or (iii) relieving the physiological change and/or pathological condition, i.e., causing regression of a disease or disorder characterized by gap junction dysfunction and/or malfunction. For example, in an aspect, treating a disease or disorder can reduce the severity of an established a disease or disorder in a subject by 1%-100% as compared to a control (such as, for example, an individual not having a disease or disorder characterized by gap junction dysfunction and/or malfunction). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as a disease or disorder characterized by gap junction dysfunction and/or malfunction). For example, treating a disease or a disorder can reduce one or more symptoms of a disease or disorder in a subject by 1%-100% as compared to a control (such as, for example, an individual not having a disease or disorder characterized by gap junction dysfunction and/or malfunction). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., gap junction dysfunction and/or malfunction). It is understood that treatment does not necessarily refer to a cure or complete ablation or eradication of a disease or disorder characterized by gap junction dysfunction and/or malfunction. However, in an aspect, treatment can refer to a cure or complete ablation or eradication of a disease or a disorder or a condition (such as gap junction dysfunction and/or malfunction).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing gap junction dysfunction and/or malfunction or the worsening of gap junction dysfunction and/or malfunction is intended. The words “prevent” and “preventing” and “prevention” also refer to prophylactic or preventative measures for protecting or precluding a subject (e.g., an individual) not having gap junction dysfunction and/or malfunction or a given gap junction dysfunction and/or malfunction-related complication from progressing to that complication.

As used herein, the terms “administering” and “administration” refer to any method of providing one or more of the disclosed compositions. Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can also include hepatic intra-arterial administration or administration through the hepatic portal vein (HPV). Administration of a disclosed composition, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route. In an aspect, a disclosed composition, a disclosed pharmaceutical formulation, or any combination thereof can be concurrently and/or serially administered to a subject via multiple routes of administration. For example, in an aspect, administering a disclosed composition, a disclosed pharmaceutical formulation, or any combination thereof can comprise intravenous administration and intra-cistern magna (ICM) administration. In an aspect, administering a disclosed composition, a disclosed pharmaceutical formulation, or any combination thereof can comprise IV administration and intrathecal (ITH) administration. Various combinations of administration are known to the skilled person.

By “determining the amount” is meant both an absolute quantification of a particular analyte (e.g., fluorescence) or a determination of the relative abundance of a particular analyte (e.g., a dual-labeled connexosome). The phrase includes both direct or indirect measurements of abundance or both.

As used herein, “modifying the method” can comprise modifying or changing one or more features or aspects of one or more steps of a disclosed method. In an aspect, a method can be altered by changing the amount of one or more of the disclosed compositions used in a disclosed method, or by changing the frequency of administration of one or more disclosed compositions in a disclosed method, by changing the duration of time that one or more disclosed compositions is administered in a disclosed method, or by substituting for one or more of the disclosed components and/or reagents with a similar or equivalent component and/or reagent.

As used herein, “concurrently” means (1) simultaneously in time, or (2) at different times during a common treatment schedule.

The term “contacting” as used herein refers to bringing one or more of the disclosed compositions together with a target area or intended target area in such a manner that the disclosed compositions can exert an effect on the intended target or targeted area either directly or indirectly. A target area or intended target area can be one or more cells or one or more connexosome, one or more connexin proteins, one or more disclosed isolated nucleic acid molecules and/or sequences, one or more disclosed cells, or any combination thereof. In an aspect, a target area or intended target area can be any cell or any organ infected by a disease or disorder (such as a cell or organ characterized by gap junction dysfunction and/or malfunction). In an aspect, a target area or intended target area can be any organ, tissue, or cells that are affected by a disease or disorder characterized by gap junction dysfunction and/or malfunction).

As used herein, “determining” can refer to measuring or ascertaining the presence and severity of a disease or disorder, such as, for example, characterized by gap junction dysfunction and/or malfunction. “Determining” can refer to measuring or ascertaining fluorescence. “Determining” can refer to measuring or ascertaining gap junction formation, or measuring or ascertaining docking interactions between connexins, or measuring or ascertaining gap junction hemichannel docking.

Methods and techniques used to determine the presence and/or severity of a disease or disorder characterized by gap junction dysfunction and/or malfunction are typically known to the medical arts. For example, the art is familiar with the ways to identify and/or diagnose the presence, severity, or both of a disease or disorder characterized by gap junction dysfunction and/or malfunction. Methods can be based on objective and/or subjective means.

As used herein, “effective amount” and “amount effective” can refer to an amount that is sufficient to achieve the desired result such as, for example, the treatment and/or prevention of a disease or disorder characterized by gap junction dysfunction and/or malfunction. As used herein, the terms “effective amount” and “amount effective” can refer to an amount that is sufficient to achieve the desired an effect on an undesired condition (e.g., a disease or disorder characterized by gap junction dysfunction and/or malfunction). For example, a “therapeutically effective amount” refers to an amount that is sufficient to achieve the desired therapeutic result or to have an effect on undesired symptoms, but is generally insufficient to cause adverse side effects.

In an aspect, “therapeutically effective amount” means an amount of the disclosed composition that (i) treats a disease or disorder characterized by gap junction dysfunction and/or malfunction, (ii) attenuates, ameliorates, or eliminates one or more symptoms associated with a disease or disorder characterized by gap junction dysfunction and/or malfunction, or (iii) delays the onset of one or more symptoms of a disease or disorder characterized by gap junction dysfunction and/or malfunction. The specific therapeutically effective dose level for any particular patient will depend upon a variety of factors including the disease or disorder characterized by gap junction dysfunction and/or malfunction being treated; the disclosed compositions employed; the disclosed methods employed; the age, body weight, general health, sex and diet of the patient; the time of administration; the route of administration; the rate of excretion of the disclosed compositions employed; the duration of the treatment; drugs used in combination or coincidental with the disclosed compositions employed, and other like factors well known in the medical arts. For example, it is well within the skill of the art to start doses of the disclosed compositions at levels lower than those required to achieve the desired therapeutic effect and to gradually increase the dosage until the desired effect is achieved. If desired, then the effective daily dose can be divided into multiple doses for purposes of administration. Consequently, a single dose of the disclosed compositions, disclosed pharmaceutical formulations, disclosed therapeutic agents, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a sign or symptom associated with a disease or disorder characterized by gap junction dysfunction and/or malfunction.

As used herein, the term “package insert” is used to refer to instructions customarily included in commercial packages of therapeutic products, that contain information about the indications, usage, dosage, administration, contraindications and/or warnings concerning the use of such therapeutic products.

Disclosed are the components to be used to prepare the disclosed compositions, disclosed pharmaceutical formulations, disclosed therapeutic agents, or a combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### C. Compositions for Use in the Disclosed Methods

### 1. Connexin Proteins

Disclosed herein is a connexin protein. Disclosed herein are connexin proteins.

In an aspect, a disclosed connexin protein can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, or CX62, or a fragment thereof.

In an aspect, a disclosed connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof.

In an aspect, a disclosed connexin protein can comprise the sequence set forth in any one of SEQ ID NO:01-SEQ ID NO:21, or a fragment thereof. In an aspect, a disclosed connexin protein can comprise the sequence set forth in any sequence provided in Table 5.

In an aspect, a disclosed connexin protein can be encoded by any one of SEQ ID NO:22-SEQ ID NO:42, or a fragment thereof. In an aspect, a disclosed connexin protein can be encoded by any sequence set forth in Table 6.

Disclosed herein is a connexin protein having one or more mutations. Disclosed herein are connexin proteins having one or more mutations.

In an aspect, a disclosed connexin protein can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, or CX62, or a fragment thereof, and which sequence comprises one or more mutations. In an aspect, a disclosed connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof and which sequence comprises one or more mutations. In an aspect, a disclosed connexin protein can comprise the sequence set forth in any one of SEQ ID NO:01-SEQ ID NO:21, or a fragment thereof. In an aspect, a disclosed connexin protein can comprise the sequence set forth in any sequence provided in Table 5.

In an aspect, a disclosed connexin protein can be encoded by any one of SEQ ID NO:22-SEQ ID NO:42, or a fragment thereof and which sequence comprises one or more mutations. In an aspect, a disclosed connexin protein can be encoded by any sequence set forth in Table 6.

In an aspect, a disclosed connexin protein can comprise one or more mutations. In an aspect, a disclosed mutation can affect a transmembrane helix, an extracellular loop, a cytoplasmic loop, an intracellular amino terminal, or an intracellular carboxy terminal.

In an aspect, a disclosed connexin protein can comprise a label and/or a tag. In an aspect, a disclosed connexin protein can comprise a fluorescent label and/or a tag. In an aspect, a disclosed connexin protein can comprise a carboxy-terminal label and/or tag or an amino-terminal label and/or tag. In an aspect, a disclosed connexin protein can comprise a carboxy-terminal fluorescent label and/or tag or an amino-terminal fluorescent label and/or tag. In an aspect, a disclosed connexin protein can comprise a fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

Disclosed herein is a fluorescently labeled connexin protein. In an aspect, a disclosed connexin protein can comprise a fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

Disclosed herein is a fluorescently labeled connexin construct. In an aspect, a disclosed construct can comprise a carboxy-terminal fluorescent label or an amino-terminal tag fluorescent label, or both.

Disclosed herein are docked hemichannels comprising a first disclosed connexin and a second disclosed connexin. Disclosed herein are docked hemichannels comprising a first labeled connexin and a second labeled connexin. Disclosed herein are hemichannel combinations comprising a first labeled connexin and a second labeled connexin. Disclosed herein is a labeled connexosome comprising a first disclosed connexin and a second disclosed connexin. In an aspect, disclosed hemichannel combinations include the combinations set forth below in Table 2.

TABLE 2
Disclosed Hemichannel Combinations.
Hemichannel Combinations

| | | | | |
|---|---|---|---|---|
| CX26/CX26 | CX43/CX26 | CX32/CX26 | CX40/CX26 | CX37/CX26 |
| CX26/CX43 | CX43/CX43 | CX32/CX43 | CX40/CX43 | CX37/CX43 |
| CX26/CX30 | CX43/CX30 | CX32/CX30 | CX40/CX30 | CX37/CX30 |
| CX26/CX32 | CX43/CX32 | CX32/CX32 | CX40/CX32 | CX37/CX32 |
| CX26/CX31 | CX43/CX31 | CX32/CX31 | CX40/CX31 | CX37/CX31 |
| CX26/CX40 | CX43/CX40 | CX32/CX40 | CX40/CX40 | CX37/CX40 |
| CX26/CX37 | CX43/CX37 | CX32/CX37 | CX40/CX37 | CX37/CX37 |
| CX26/CX46 | CX43/CX46 | CX32/CX46 | CX40/CX46 | CX37/CX46 |
| CX26/CX50 | CX43/CX50 | CX32/CX50 | CX40/CX50 | CX37/CX50 |
| CX26/CX30.3 | CX43/CX30.3 | CX32/CX30.3 | CX40/CX30.3 | CX37/CX30.3 |
| CX26/CX45 | CX43/CX45 | CX32/CX45 | CX40/CX45 | CX37/CX45 |
| CX26/CX31.1 | CX43/CX31.1 | CX32/CX31.1 | CX40/CX31.1 | CX37/CX31.1 |
| CX26/CX36 | CX43/CX36 | CX32/CX36 | CX40/CX36 | CX37/CX36 |
| CX26/CX47 | CX43/CX47 | CX32/CX47 | CX40/CX47 | CX37/CX47 |
| CX26/CX30.2 | CX43/CX30.2 | CX32/CX30.2 | CX40/CX30.2 | CX37/CX30.2 |
| CX26/CX59 | CX43/CX59 | CX32/CX59 | CX40/CX59 | CX37/CX59 |
| CX26/CX62 | CX43/CX62 | CX32/CX62 | CX40/CX62 | CX37/CX62 |
| CX26/CX25 | CX43/CX25 | CX32/CX25 | CX40/CX25 | CX37/CX25 |
| CX26/CX31.9 | CX43/CX31.9 | CX32/CX31.9 | CX40/CX31.9 | CX37/CX31.9 |
| CX26/CX40.1 | CX43/CX40.1 | CX32/CX40.1 | CX40/CX40.1 | CX37/CX40.1 |
| CX26/CX23 | CX43/CX23 | CX32/CX23 | CX40/CX23 | CX37/CX23 |
| CX30/CX26 | CX31/CX26 | CX46/CX26 | CX50/CX26 | CX50/CX26 |
| CX30/CX43 | CX31/CX43 | CX46/CX43 | CX50/CX43 | CX50/CX43 |
| CX30/CX30 | CX31/CX30 | CX46/CX30 | CX50/CX30 | CX50/CX30 |
| CX30/CX32 | CX31/CX32 | CX46/CX32 | CX50/CX32 | CX50/CX32 |
| CX30/CX31 | CX31/CX31 | CX46/CX31 | CX50/CX31 | CX50/CX31 |
| CX30/CX40 | CX31/CX40 | CX46/CX40 | CX50/CX40 | CX50/CX40 |
| CX30/CX37 | CX31/CX37 | CX46/CX37 | CX50/CX37 | CX50/CX37 |
| CX30/CX46 | CX31/CX46 | CX46/CX46 | CX50/CX46 | CX50/CX46 |
| CX30/CX50 | CX31/CX50 | CX46/CX50 | CX50/CX50 | CX50/CX50 |
| CX30/CX30.3 | CX31/CX30.3 | CX46/CX30.3 | CX50/CX30.3 | CX50/CX30.3 |
| CX30/CX45 | CX31/CX45 | CX46/CX45 | CX50/CX45 | CX50/CX45 |
| CX30/CX31.1 | CX31/CX31.1 | CX46/CX31.1 | CX50/CX31.1 | CX50/CX31.1 |
| CX30/CX36 | CX31/CX36 | CX46/CX36 | CX50/CX36 | CX50/CX36 |
| CX30/CX47 | CX31/CX47 | CX46/CX47 | CX50/CX47 | CX50/CX47 |
| CX30/CX30.2 | CX31/CX30.2 | CX46/CX30.2 | CX50/CX30.2 | CX50/CX30.2 |
| CX30/CX59 | CX31/CX59 | CX46/CX59 | CX50/CX59 | CX50/CX59 |
| CX30/CX62 | CX31/CX62 | CX46/CX62 | CX50/CX62 | CX50/CX62 |
| CX30/CX25 | CX31/CX25 | CX46/CX25 | CX50/CX25 | CX50/CX25 |
| CX30/CX31.9 | CX31/CX31.9 | CX46/CX31.9 | CX50/CX31.9 | CX50/CX31.9 |
| CX30/CX40.1 | CX31/CX40.1 | CX46/CX40.1 | CX50/CX40.1 | CX50/CX40.1 |
| CX30/CX23 | CX31/CX23 | CX46/CX23 | CX50/CX23 | CX50/CX23 |
| CX45/CX26 | CX31.1/CX26 | CX36/CX26 | CX47/CX26 | CX30.2/CX26 |
| CX45/CX43 | CX31.1/CX43 | CX36/CX43 | CX47/CX43 | CX30.2/CX43 |
| CX45/CX30 | CX31.1/CX30 | CX36/CX30 | CX47/CX30 | CX30.2/CX30 |
| CX45/CX32 | CX31.1/CX32 | CX36/CX32 | CX47/CX32 | CX30.2/CX32 |
| CX45/CX31 | CX31.1/CX31 | CX36/CX31 | CX47/CX31 | CX30.2/CX31 |
| CX45/CX40 | CX31.1/CX40 | CX36/CX40 | CX47/CX40 | CX30.2/CX40 |
| CX45/CX37 | CX31.1/CX37 | CX36/CX37 | CX47/CX37 | CX30.2/CX37 |
| CX45/CX46 | CX31.1/CX46 | CX36/CX46 | CX47/CX46 | CX30.2/CX46 |
| CX45/CX50 | CX31.1/CX50 | CX36/CX50 | CX47/CX50 | CX30.2/CX50 |
| CX45/CX30.3 | CX31.1/CX30.3 | CX36/CX30.3 | CX47/CX30.3 | CX30.2/CX30.3 |
| CX45/CX45 | CX31.1/CX45 | CX36/CX45 | CX47/CX45 | CX30.2/CX45 |
| CX45/CX31.1 | CX31.1/CX31.1 | CX36/CX31.1 | CX47/CX31.1 | CX30.2/CX31.1 |
| CX45/CX36 | CX31.1/CX36 | CX36/CX36 | CX47/CX36 | CX30.2/CX36 |
| CX45/CX47 | CX31.1/CX47 | CX36/CX47 | CX47/CX47 | CX30.2/CX47 |
| CX45/CX30.2 | CX31.1/CX30.2 | CX36/CX30.2 | CX47/CX30.2 | CX30.2/CX30.2 |
| CX45/CX59 | CX31.1/CX59 | CX36/CX59 | CX47/CX59 | CX30.2/CX59 |
| CX45/CX62 | CX31.1/CX62 | CX36/CX62 | CX47/CX62 | CX30.2/CX62 |
| CX45/CX25 | CX31.1/CX25 | CX36/CX25 | CX47/CX25 | CX30.2/CX25 |
| CX45/CX31.9 | CX31.1/CX31.9 | CX36/CX31.9 | CX47/CX31.9 | CX30.2/CX31.9 |
| CX45/CX40.1 | CX31.1/CX40.1 | CX36/CX40.1 | CX47/CX40.1 | CX30.2/CX40.1 |
| CX45/CX23 | CX31.1/CX23 | CX36/CX23 | CX47/CX23 | CX30.2/CX23 |
| CX62/CX26 | CX25/CX26 | CX31.9/CX26 | CX40.1/CX26 | CX23/CX26 |
| CX62/CX43 | CX25/CX43 | CX31.9/CX43 | CX40.1/CX43 | CX23/CX43 |
| CX62/CX30 | CX25/CX30 | CX31.9/CX30 | CX40.1/CX30 | CX23/CX30 |
| CX62/CX32 | CX25/CX32 | CX31.9/CX32 | CX40.1/CX32 | CX23/CX32 |
| CX62/CX31 | CX25/CX31 | CX31.9/CX31 | CX40.1/CX31 | CX23/CX31 |
| CX62/CX40 | CX25/CX40 | CX31.9/CX40 | CX40.1/CX40 | CX23/CX40 |
| CX62/CX37 | CX25/CX37 | CX31.9/CX37 | CX40.1/CX37 | CX23/CX37 |
| CX62/CX46 | CX25/CX46 | CX31.9/CX46 | CX40.1/CX46 | CX23/CX46 |
| CX62/CX50 | CX25/CX50 | CX31.9/CX50 | CX40.1/CX50 | CX23/CX50 |
| CX62/CX30.3 | CX25/CX30.3 | CX31.9/CX30.3 | CX40.1/CX30.3 | CX23/CX30.3 |
| CX62/CX45 | CX25/CX45 | CX31.9/CX45 | CX40.1/CX45 | CX23/CX45 |
| CX62/CX31.1 | CX25/CX31.1 | CX31.9/CX31.1 | CX40.1/CX31.1 | CX23/CX31.1 |
| CX62/CX36 | CX25/CX36 | CX31.9/CX36 | CX40.1/CX36 | CX23/CX36 |
| CX62/CX47 | CX25/CX47 | CX31.9/CX47 | CX40.1/CX47 | CX23/CX47 |
| CX62/CX30.2 | CX25/CX30.2 | CX31.9/CX30.2 | CX40.1/CX30.2 | CX23/CX30.2 |
| CX62/CX59 | CX25/CX59 | CX31.9/CX59 | CX40.1/CX59 | CX23/CX59 |
| CX62/CX62 | CX25/CX62 | CX31.9/CX62 | CX40.1/CX62 | CX23/CX62 |
| CX62/CX25 | CX25/CX25 | CX31.9/CX25 | CX40.1/CX25 | CX23/CX25 |
| CX62/CX31.9 | CX25/CX31.9 | CX31.9/CX31.9 | CX40.1/CX31.9 | CX23/CX31.9 |
| CX62/CX40.1 | CX25/CX40.1 | CX31.9/CX40.1 | CX40.1/CX40.1 | CX23/CX40.1 |
| CX62/CX23 | CX25/CX23 | CX31.9/CX23 | CX40.1/CX23 | CX23/CX23 |
| CX59/CX26 | CX59/CX31 | CX59/CX50 | CX59/CX36 | CX59/CX62 |
| CX59/CX43 | CX59/CX40 | CX59/CX30.3 | CX59/CX47 | CX59/CX25 |
| CX59/CX30 | CX59/CX37 | CX59/CX45 | CX59/CX30.2 | CX59/CX31.9 |
| CX59/CX32 | CX59/CX46 | CX59/CX31.1 | CX59/CX59 | CX59/CX40.1 |
| CX59/CX23 | | | | |

### 2. Connexin Nucleic Add Molecules

Disclosed herein is an isolated nucleic acid molecule comprising a sequence that encodes the sequence set forth in any one of SEQ ID NO:01-SEQ ID NO:21, or a fragment thereof. Disclosed herein is an isolated nucleic acid molecule comprising a sequence that encodes the sequence set forth in any one sequence provided in Table 5. Disclosed herein is an isolated nucleic acid molecule comprising the sequence set forth in any of SEQ ID NO:22 to SEQ ID NO:42, or a fragment thereof. Disclosed herein is an isolated nucleic acid molecule comprising any sequence set forth in Table 6. Disclosed herein is an isolated nucleic acid molecule comprising a sequence that encodes the sequence set forth in any one of SEQ ID NO:01-SEQ ID NO:21, or a fragment thereof, and which sequence comprises one or more mutations. Disclosed herein is an isolated nucleic acid molecule comprising the sequence set forth in any of SEQ ID NO:22 to SEQ ID NO:42, or a fragment thereof, and which sequence comprises one or more mutations.

Disclosed herein is an isolated nucleic acid molecule comprising a sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof. Disclosed herein is an isolated nucleic acid molecule comprising a sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof. Disclosed herein is an isolated nucleic acid molecule comprising a sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof. Disclosed herein is an isolated nucleic acid molecule comprising a sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof, and which sequence comprises one or more mutations. Disclosed herein is an isolated nucleic acid molecule comprising a sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof and which sequence comprises one or more mutations. Disclosed herein is an isolated nucleic acid molecule comprising a sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, and which sequence comprises one or more mutations.

In an aspect, a disclosed mutation can affect a transmembrane helix, an extracellular loop, a cytoplasmic loop, an intracellular amino terminal, or an intracellular carboxy terminal of a disclosed connexin protein. In an aspect, a disclosed nucleic acid sequence can be codon-optimized, for example, for expression in human cells.

Disclosed herein is an isolated nucleic acid molecule comprising a sequence that encodes the sequence set forth in any one of SEQ ID NO:01-SEQ ID NO:21, or a fragment thereof, or a mutant thereof, and a sequence encoding a tag and/or label.

Disclosed herein is an isolated nucleic acid molecule comprising a sequence that encodes the sequence set forth in any one sequence in Table 5, and a sequence encoding a tag and/or label.

Disclosed herein is an isolated nucleic acid molecule comprising a sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof, or a mutant thereof, and a sequence encoding a tag and/or label. Disclosed herein is an isolated nucleic acid molecule comprising a sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof, or a mutant thereof, and a sequence encoding a tag and/or label. Disclosed herein is an isolated nucleic acid molecule comprising a sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, or a mutant thereof, and a sequence encoding a tag and/or label.

Disclosed herein is an isolated nucleic acid molecule comprising the sequence set forth in any one of SEQ ID NO:22 to SEQ ID NO:42, or a fragment thereof, or a mutant thereof, and a sequence encoding a tag and/or label.

In an aspect, a disclosed nucleic acid sequence can be codon-optimized, for example, for expression in human cells, and a sequence encoding a tag and/or label.

Disclosed herein is an isolated nucleic acid molecule comprising a sequence that encodes the sequence set forth in any one of SEQ ID NO:01-SEQ ID NO:21, or a fragment thereof, or a mutant thereof, and a sequence encoding a tag and/or label. Disclosed herein is an isolated nucleic acid molecule comprising a sequence that encodes the sequence set forth in any one sequence of Table 5, or a fragment thereof, or a mutant thereof, and a sequence encoding a tag and/or label.

Disclosed herein is an isolated nucleic acid molecule comprising the sequence set forth in any one of SEQ ID NO:22-SEQ ID NO:42, or a fragment thereof, or a mutant thereof, and a sequence encoding a tag and/or label.

Disclosed herein are docked hemichannels comprising a first disclosed connexin encoded by a disclosed isolated nucleic acid molecule and a second disclosed connexin encoded by a disclosed isolated nucleic acid molecule.

Disclosed herein are docked hemichannels comprising a first disclosed connexin encoded by a disclosed isolated nucleic acid molecule comprising the sequence set forth in any one of SEQ ID NO:22-SEQ ID NO:42, or a fragment thereof, or a mutant thereof, and a second disclosed connexin encoded by a disclosed isolated nucleic acid molecule comprising the sequence set forth in any one of SEQ ID NO:22-SEQ ID NO:42, or a fragment thereof, or a mutant thereof.

### 3. Cells

Disclosed herein is a cell comprising a disclosed isolated nucleic acid molecule or a disclosed plasmid. Disclosed herein are cells transfected by one or more disclosed nucleic acid molecules. In an aspect, disclosed transfected cells can comprise HEK 293FT cells. HEK 293FT cells are known to the art. In an aspect, disclosed transfected cells can comprise any human cells. In an aspect, disclosed transfected cells can comprise any cells that express connexins. In an aspect, disclosed transfected cells can comprise any cells that express connexins. Cells are known to the art.

Disclosed herein are cells transfected by any plasmid set forth in Table 3. Disclosed herein are a pair of cells, each transfected by a plasmid set forth in Table 3.

Disclosed herein are cells transfected by an isolated nucleic acid molecule comprising a nucleic acid sequence encoding the sequence set forth in any one of SEQ ID NO:01-SEQ ID NO:21, or a fragment thereof. Disclosed herein are cells transfected by an isolated nucleic acid molecule comprising a nucleic acid sequence encoding the sequence set forth in any sequence provided in Table 5.

Disclosed herein are cells transfected by an isolated nucleic acid molecule comprising a nucleic acid sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof. Disclosed herein are cells transfected by an isolated nucleic acid molecule comprising the nucleic acid sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof. Disclosed herein are cells transfected by an isolated nucleic acid molecule comprising a nucleic acid sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof.

Disclosed herein are cells transfected by an isolated nucleic acid molecule comprising the sequence set forth in any one of SEQ ID NO:22 to SEQ ID NO:42, or a fragment thereof. Disclosed herein are cells transfected by an isolated nucleic acid molecule comprising any sequence set forth in Table 6. Disclosed herein are cells transfected by an isolated nucleic acid molecule comprising a nucleic acid sequence encoding the sequence set forth in any one of SEQ ID NO:01-SEQ ID NO:21, or a fragment thereof, and which sequence comprises one or more mutations.

Disclosed herein are cells transfected by an isolated nucleic acid molecule comprising a nucleic acid sequence that encodes the sequence set forth in UniKBProt sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, or A6NN92, or a fragment thereof and which sequence comprises one or more mutations. Disclosed herein are cells transfected by an isolated nucleic acid molecule comprising the nucleic acid sequence set forth in NCBI Entrez Gene ID #2072, 2697, 2700, 2701, 2703, 2705, 2706, 2707, 2709, 10052, 10804, 57165, 57369, 81025, 84694, 125111, 127534, 219770, 349149, 375519, or 100126572, or a fragment thereof, and which sequence comprises one or more mutations.

Disclosed herein are cells transfected by an isolated nucleic acid molecule comprising a nucleic acid sequence corresponding to GJB2, GJA1, GJB6, GJB1, GJB3, GJA5, GJA4, GJA3, GJA8, GJB4, GJC1, GJB5, GJD2, GJC2, GJC3, GJA9, GJA10, GJB7, GJD3, GJD4, or GJE1, or a fragment thereof, and which sequence comprises one or more mutations.

Disclosed herein are cells transfected by an isolated nucleic acid molecule comprising the sequence set forth in any one of SEQ ID NO:22 to SEQ ID NO:42, or a fragment thereof, and which sequence comprises one or more mutations. Disclosed herein are cells transfected by an isolated nucleic acid molecule comprising any sequence set forth in Table 6, and which sequence comprises one or more mutations.

In an aspect, disclosed cells can comprise cells harvested and/or obtained from a subject. In an aspect, disclosed cells can comprise cells harvested and/or obtained from a subject suspected of having or diagnosed with a disease or disorder such as, for example, a disease or disorder characterized by gap junction dysfunction and/or gap junction pathophysiology.

Techniques to achieve transfection are known to the art and using transfected cells are known to the art.

### 4. Plasmids

Disclosed herein is a plasmid used in a disclosed method. Disclosed herein is a plasmid comprising one or more disclosed isolated nucleic acid molecules. Disclosed here are the plasmids of Table 3, which can be used in a disclosed method. Disclosed here are a pair of plasmids that can be used in a disclosed method, wherein the pair of plasmids are set forth in Table 3.

For example, in an aspect, a disclosed plasmid can comprise an isolated nucleic acid molecule comprising the sequence set forth in any one of SEQ ID NO:22-SEQ ID NO:42, or a fragment thereof. In an aspect, a disclosed plasmid can comprise an isolated nucleic acid molecule encoding the sequence set forth in any one of SEQ ID NO:01-SEQ ID NO:21, or a fragment thereof. For example, in an aspect, a disclosed plasmid can comprise an isolated nucleic acid molecule comprising the sequence set forth in any one of SEQ ID NO:22-SEQ ID NO:42, or a fragment thereof, and a nucleic acid sequence encoding a fluorescent label and/or tag. In an aspect, a disclosed plasmid can comprise an isolated nucleic acid molecule encoding the sequence set forth in any one of SEQ ID NO:01-SEQ ID NO:21, or a fragment thereof, and a nucleic acid sequence encoding a fluorescent label and/or tag. In an aspect, a disclosed plasmid can comprise an isolated nucleic acid molecule comprising the sequence set forth in any one of SEQ ID NO:22-SEQ ID NO:42, or a fragment thereof, and which sequence comprises one or more mutations. In an aspect, a disclosed plasmid can comprise an isolated nucleic acid molecule encoding the sequence set forth in any one of SEQ ID NO:01-SEQ ID NO:21, or a fragment thereof, and which sequence comprises one or more mutations. For example, in an aspect, a disclosed plasmid can comprise an isolated nucleic acid molecule comprising the sequence set forth in any one of SEQ ID NO:22-SEQ ID NO:42, or a fragment thereof, and which sequence comprises one or more mutations, and a nucleic acid sequence encoding a fluorescent label and/or tag. In an aspect, a disclosed plasmid can comprise an isolated nucleic acid molecule encoding the sequence set forth in any one of SEQ ID NO:01-SEQ ID NO:21, or a fragment thereof, and which sequence comprises one or more mutations, and a nucleic acid sequence encoding a fluorescent label and/or tag.

In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. The art if fam Fluorophores and fluorescent labels are known in the art.

Disclosed herein is a plasmid comprising the sequence set forth in any one of SEQ ID NO:43-SEQ ID NO:86 or a fragment thereof. Disclosed herein is a plasmid comprising the sequence set forth in any one sequence set forth in Table 3. Plasmids and using plasmids are known to the art.

TABLE 3
Disclosed Plasmids.

| SEQ ID NO: | Plasmid Description |
|---|---|
| 43 | Cx23-mEmeraldN1 |
| 44 | Cx25-mEmeraldN1 |
| 45 | Cx26-mEmeraldN1 |
| 46 | Cx30-mEmeraldN1 |
| 47 | Cx30.3-mEmeraldN1 |
| 48 | Cx31-mEmeraldN1 |
| 49 | Cx31.1-mEmeraldN1 |
| 50 | Cx31.3-mEmeraldN1 |
| 51 | Cx31.9-mEmeraldN1 |
| 52 | Cx32-mEmeraldN1 |
| 53 | Cx36-mEmeraldN1 |
| 54 | Cx37-mEmeraldN1 |
| 55 | Cx40-mEmeraldN1 |
| 56 | Cx40.1-mEmeraldN1 |
| 57 | Cx43-mEmeraldN1 |
| 58 | Cx45-mEmeraldN1 |
| 59 | Cx46-mEmeraldN1 |
| 60 | Cx47-mEmeraldN1 |
| 61 | Cx50-mEmeraldN1 |
| 62 | Cx59-mEmeraldN1 |
| 63 | Cx62-mEmeraldN1 |
| 64 | Cx43-ELs-Cx26-mEmeraldN1 |
| 65 | Cx23-iRFP670 |
| 66 | Cx25-iRFP670 |
| 67 | Cx26-iRFP670 |
| 68 | Cx30-iRFP670 |
| 69 | Cx30.3-iRFP670 |
| 70 | Cx31-iRFP670 |
| 71 | Cx31.1-iRFP670 |
| 72 | Cx31.3 -iRFP670 |
| 73 | Cx31.9-iRFP670 |
| 74 | Cx32-iRFP670 |
| 75 | Cx36-iRFP670 |
| 76 | Cx37-iRFP670 |
| 77 | Cx40-iRFP670 |
| 78 | Cx40.1-iRFP670 |
| 79 | Cx43-iRFP670 |
| 80 | Cx45-iRFP670 |
| 81 | Cx46-iRFP670 |
| 82 | Cx47-iRFP670 |
| 83 | Cx50-iRFP670 |
| 84 | Cx59-iRFP670 |
| 85 | Cx62-iRFP670 |
| 86 | Cx43-mAppleN1 |

### 5. Kits

Disclosed herein is a kit comprising one or more disclosed isolated nucleic acid molecules, one or more disclosed proteins, one or more disclosed cells, or any combination thereof. Disclosed herein is a kit comprising a disclosed connexin protein. Disclosed herein is a kit comprising a disclosed connexin protein comprising a label and/or a tag. Disclosed herein is a kit comprising one or more disclosed cells. Disclosed herein is a kit comprising one or more disclosed isolated nucleic acid molecules. Disclosed herein is a kit comprising one or more disclosed cells transfected with a nucleic acid molecule comprising the sequence set forth in any one of SEQ ID NO:22-SEQ ID NO:42, or a fragment thereof. Disclosed herein is a kit comprising one or more disclosed cells transfected with a nucleic acid molecule encoding a connexin having the sequence set forth in any one of SEQ ID NO:01-SEQ ID NO:21, or a fragment thereof. Disclosed herein is a kit comprising one or more disclosed cells transfected with a nucleic acid molecule comprising the sequence set forth in any one of SEQ ID NO:22-SEQ ID NO:42, or a fragment thereof, and which sequence comprises one or more mutations. Disclosed herein is a kit comprising one or more disclosed cells transfected with a nucleic acid molecule encoding a connexin having the sequence set forth in any one of SEQ ID NO:01-SEQ ID NO:21, or a fragment thereof, and which sequence comprises one or more mutations.

Disclosed herein is a kit comprising one or more disclosed compositions and/or components and/or agents that can be used in any disclosed method.

In an aspect of a disclosed kit, a disclosed fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect of a disclosed kit, a disclosed fluorescent label or disclosed fluorophore can comprise any fluorescent label or fluorophore that is amendable to analysis via flow cytometry. In an aspect of a disclosed kit, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed kit can comprise at least two components constituting the kit. Together, the components constitute a functional unit for a given purpose (such as, for example, performing any aspect of a disclosed method including preparing the components used in a disclosed method). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container may be an intravenous solution bag or a vial having a stopper pierceable by a hypodermic injection needle). The label or package insert can indicate that a disclosed composition, a disclosed connexin, a disclosed nucleic acid molecule, a disclosed cell, or a combination thereof, can be used in a disclosed method. A kit can comprise additional components necessary for administration such as, for example, other buffers, diluents, filters, needles, and syringes. In an aspect, a disclosed kit can be used to evaluate gap junction formation, interrogate the docking interactions between connexin, perform high-throughput quantification of gap junction hemichannel docketing, or any combination thereof.

In an aspect, a disclosed kit can comprise one or more disclosed plasmids, such as, for example, those plasmids identified in Table 3. In an aspect, a disclosed kit can comprise one or more disclosed primers, such as, for example, those plasmids identified in Table 4. In an aspect, a disclosed kit can comprise one or more populations of cells, each population transfected by a plasmids identified in Table 4.

### B. Methods

### 1. Methods of Evaluating Gap Junction Formation

Disclosed herein is a method of evaluating gap junction formation, the method comprising performing flow enabled tracking of connexosomes in HEK293T cells (FETCH).

In an aspect, performing FETCH can comprises (i) generating fluorescent connexosomes, and (ii) using flow cytometry to track the fluorescent connexosomes in one or more cell samples. In an aspect, performing FETCH can comprise employing one or more plasmids listed in Table 3.

In an aspect, generating one or more fluorescent connexosomes can comprise transfecting a first population of cells with a first fluorescently labeled, C-terminally fused connexin construct; and transfecting a second population of cells with a second fluorescently-labeled, C-terminally fused connexin construct.

In an aspect, a disclosed method of evaluating gap junction formation can employ one or more plasmids listed in Table 3. In an aspect, one or more disclosed plasmids can comprise any pair of fluorescent labels or fluorophores that is amendable to analysis via flow cytometry and have excitation and emission spectra that can be isolated or separated from each other, thereby enabling the interrogation of the docking interactions.

In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed method can further comprise incubating the transfected populations of cells. In an aspect of a disclosed method, incubating the transfected populations of cells can comprise about 10 hours to about 24 hours. For example, in an aspect, incubating can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, or about 24 hours. In an aspect, incubating can comprise less than 10 hrs or more than 24 hrs. In an aspect, incubating the transfected populations of cells can comprises about 16 hours to about 18 hours.

In an aspect, a disclosed method can further comprise trypsinizing the incubated and transfected population of cells. Trypsinization, methods of trypsinization, and agents for trypsinizing are known to the art.

In an aspect, a disclosed method can further comprise incubating the trypsinized first population of cells with the trypsinized second population of cells.

In an aspect of a disclosed method, incubating the combined populations of cells can comprise about 10 hours to about 30 hours. For example, in an aspect, incubating the combined populations of cells can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, about 24 hrs, about 25 hrs, about 26 hrs, about 27 hrs, about 28 hrs, about 29 hrs, or about 30 hrs. In an aspect, incubating the combined populations of cells can comprise less than 10 hrs or more than 30 hrs. In an aspect, incubating the combined populations of cells can comprise about 20 hrs. In an aspect of a disclosed method, incubating the combined populations of cells can continue to hyperdensity and/or over-confluency.

In an aspect, a disclosed method can comprise trypsinizing the incubated combined population of cells, resuspending the trypsinized cells, and fixing the resuspended cells.

In an aspect, resuspending the trypsinized cells can comprise PBS and DNase. In an aspect of a disclosed method, fixing the resuspended cells can comprise paraformaldehyde. Methods of fixation and agents for fixation are known to the art.

In an aspect of a disclosed method, during flow cytometry, the cells can be analyzed in two selection gates prior to fluorescence evaluation. In an aspect, analyzing the cells in two selection gates can comprises (i) identifying cells by evaluating sample forward vs. side scatter area; and (ii) identifying single cells by evaluating cells that maintained a linear correlation of forward scatter height to forward scatter area.

In an aspect, incubating the combined populations of cells can comprise further comprising generating one or more fluorescently-labeled, C-terminally fused connexin constructs. Isolated nucleic acid molecules comprising a nucleic acid sequence encoding a connexin protein are disclosed herein.

In an aspect, a disclosed first fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof. In an aspect, a disclosed second fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof.

In an aspect, a disclosed first connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof. In an aspect, a disclosed second connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof.

In an aspect, a disclosed first connexin protein can comprise the sequence set forth in any one of SEQ ID NO:01-SEQ ID NO:21, or a fragment thereof. In an aspect, a disclosed first connexin protein can comprise the sequence set forth in any sequence provided in Table 5. In an aspect, a disclosed second connexin protein can comprise the sequence set forth in any one of SEQ ID NO:01-SEQ ID NO:21, or a fragment thereof. In an aspect, a disclosed second connexin protein can comprise the sequence set forth in any sequence provided in Table 5. In an aspect, a disclosed first connexin protein can be encoded by the sequence set forth in any one of SEQ ID NO:22-SEQ ID NO:42, or a fragment thereof, or a mutant thereof. In an aspect, a disclosed second connexin protein can be encoded by the sequence set forth in any one of SEQ ID NO:22-SEQ ID NO:42, or a fragment thereof, or a mutant thereof. In an aspect, a disclosed connexin construct can comprise a connexin suspected of having an etiological influence in a disease or disorder. In an aspect, a disclosed connexin construct can comprise a connexin confirmed to have an etiological influence in a disease or disorder.

In an aspect, flow cytometry can generate a fluorescent profile for each cell sample or each population of cells.

In an aspect, a disclosed fluorescence profile can comprise 4 quadrants. In an aspect, disclosed quadrants can comprise Q1, Q2, Q3, and Q4. In an aspect, a disclosed Q1 can represent a population of cells with the first fluorescently-labeled, C-terminally fused connexin construct. In an aspect, a disclosed Q2 can represent a population of cells expressing both the first fluorescently-labeled, C-terminally fused connexin construct and the second fluorescently-labeled C-terminally fused connexin construct. In an aspect, a disclosed Q3 can represent a population of cells with the second fluorescently-labeled C-terminally fused connexin construct. In an aspect, a disclosed Q4 can represent untransfected cells in either the first population of cells or the second populations of cells.

In an aspect, a disclosed method of evaluating gap junction formation can further comprise establishing a FETCH score. In an aspect, a disclosed FETCH score can be a positive FETCH score or a negative FETCH score. In an aspect, a disclosed positive FETCH score can indicate a docking event. In an aspect, a disclosed negative FETCH score can indicate a non-docking event. In an aspect, a disclosed negative FETCH score can indicate dysfunctional gap junction intercellular communication. In an aspect, a disclosed FETCH score can comprise the proportion of dual colored-cells compared to fluorescent cells. In an aspect, a disclosed proportion can comprise Q2/(Q1+Q2+Q3).

In an aspect, a disclosed method of evaluating gap junction formation can comprise quantifying the fluorescence exchange between cells mediated by connexosomes.

In an aspect, a disclosed method of evaluating gap junction formation can comprise confirming the relationship between the fluorescence exchange phenotype and the connexosome formation.

In an aspect, confirming the relationship between the fluorescence exchange phenotype and connexosome formation can comprise using fluorescence-activated cell sorting (FACS) to collect cells from Q2 for microscopy analysis.

In an aspect, FACS can confirm the formation of dual-labeled connexosomes.

In an aspect, a disclosed method of evaluating gap junction formation can identify homotypic docking of hemichannels. In an aspect, a disclosed method of evaluating gap junction formation can identify homotypic docking of hemichannels. In an aspect, disclosed homotypic docking can comprise docking of two hemichannels having a single connexin isoform. In an aspect, a disclosed method of evaluating gap junction formation can identify heterotypic docking of hemichannels. In an aspect, a disclosed heterotypic docking can comprise docking of two hemichannels having a different connexin isoform.

In an aspect, a disclosed method of evaluating gap junction formation can further comprise performing whole-cell, dual-patch clamp analysis on a disclosed combined population of cells.

In an aspect, a disclosed method of evaluating gap junction formation can identify variation in the expression level of one or more disclosed connexin constructs. In an aspect, a disclosed method of evaluating gap junction formation can identify variation in trafficking of one or more disclosed connexin constructs. In an aspect, a disclosed method of evaluating gap junction formation can identify variation in stability of one or more disclosed connexin constructs. In an aspect, a disclosed method of evaluating gap junction formation can identify variation in the turnover rate of one or more disclosed connexin constructs.

In an aspect, a disclosed method of evaluating gap junction formation does not quantify hemichannel binding affinity.

In an aspect, a disclosed method of evaluating gap junction formation can detect dysfunctional gap junction formation and/or communication. In an aspect, a disclosed dysfunctional gap junction formation and/or communication can be due to non-conductive hemichannels. In an aspect, a disclosed dysfunctional gap junction formation and/or communication can be due to non-formation of hemichannels.

In an aspect of a disclosed method of evaluating gap junction formation, a disclosed FETCH score can reflect variation in (i) the expression level of the connexin constructs, (ii) the trafficking of the connexin constructs, (iii) the stability of the connexin constructs, and/or (iv) the turnover rate of the connexin constructs. In an aspect, a disclosed method of evaluating gap junction formation can be a downstream indicator of connexin hemichannel docking compatibility.

In an aspect, a disclosed method of evaluating gap junction formation can be scaled for high-throughput analysis.

In an aspect, a disclosed method of evaluating gap junction formation can evaluate one or more engineered connexin constructs for one or more new docking features. For example, in an aspect, an engineered connexin construct can comprise a modified CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or any combination thereof.

In an aspect, a disclosed method of evaluating gap junction formation can further comprise interrogating the docking interactions between connexins. In an aspect, a disclosed method can further comprise performing high-throughput quantification of gap junction hemichannel docking.

Disclosed herein is method of evaluating gap junction formation, the method comprising generating fluorescent connexosomes, wherein generating one or more fluorescent connexosomes comprises transfecting a first population of cells with a first fluorescently-labeled, C-terminally fused connexin construct; transfecting a second population of cells with a second fluorescently-labeled, C-terminally fused connexin construct; incubating the transfected populations of cells; trypsinizing the incubated and transfected population of cells; incubating the trypsinized first population of cells with the trypsinized second population of cells; trypsinizing the incubated combined population of cells, resuspending the trypsinized cells, and fixing the resuspended cells; and performing flow cytometry to track the fluorescent connexosomes by quantifying the fluorescence exchange between cells mediated by connexosomes.

In an aspect, a disclosed method of evaluating gap junction formation can further comprise treating a subject having a disease or disorder characterized by gap junction dysfunction and/or malfunction. In an aspect, gap junction dysfunction and/or malfunction can be confirmed by a disclosed method.

In an aspect, a disclosed method of evaluating gap junction formation can further comprise interrogating the docking interactions between connexins. In an aspect, a disclosed method of evaluating gap junction formation can further comprise high-throughput quantification of gap junction hemichannel docking. In an aspect, a disclosed method of evaluating gap junction formation can further comprise interrogating the docking interactions between connexins and high-throughput quantification of gap junction hemichannel docking.

In an aspect, a disclosed method of evaluating gap junction formation can use a disclosed automated pipeline that can receive and process FETCH data. A disclosed automated pipeline is described infra.

### 2. Methods of Interrogating the Docking Interactions Between Connexins

Disclosed herein is a method of interrogating the docking interactions between connexins, the method comprising performing flow enabled tracking of connexosomes in HEK293T cells (FETCH).

In an aspect, performing FETCH can comprises (i) generating fluorescent connexosomes, and (ii) using flow cytometry to track the fluorescent connexosomes in one or more cell samples. In an aspect, performing FETCH can comprise employing one or more plasmids listed in Table 3.

In an aspect, generating one or more fluorescent connexosomes can comprise transfecting a first population of cells with a first fluorescently-labeled, C-terminally fused connexin construct; and transfecting a second population of cells with a second fluorescently-labeled, C-terminally fused connexin construct.

In an aspect, a disclosed method of interrogating the docking interactions can employ one or more plasmids listed in Table 3. In an aspect, one or more disclosed plasmids can comprise any pair of fluorescent labels or fluorophores that is amendable to analysis via flow cytometry and have excitation and emission spectra that can be isolated or separated from each other, thereby enabling the interrogation of the docking interactions.

In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed method can further comprise incubating the transfected populations of cells. In an aspect of a disclosed method, incubating the transfected populations of cells can comprise about 10 hours to about 24 hours. For example, in an aspect, incubating can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, or about 24 hours. In an aspect, incubating can comprise less than 10 hrs or more than 24 hrs. In an aspect, incubating the transfected populations of cells can comprises about 16 hours to about 18 hours.

In an aspect, a disclosed method can further comprise trypsinizing the incubated and transfected population of cells. Trypsinization, methods of trypsinization, and agents for trypsinizing are known to the art.

In an aspect, a disclosed method can further comprise incubating the trypsinized first population of cells with the trypsinized second population of cells. In an aspect of a disclosed method, incubating the combined populations of cells can comprise about 10 hours to about 30 hours. For example, in an aspect, incubating the combined populations of cells can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, about 24 hrs, about 25 hrs, about 26 hrs, about 27 hrs, about 28 hrs, about 29 hrs, or about 30 hrs. In an aspect, incubating the combined populations of cells can comprise less than 10 hrs or more than 30 hrs. In an aspect, incubating the combined populations of cells can comprise about 20 hrs. In an aspect of a disclosed method, incubating the combined populations of cells can continue to hyperdensity and/or over-confluency.

In an aspect, a disclosed method can comprise trypsinizing the incubated combined population of cells, resuspending the trypsinized cells, and fixing the resuspended cells. In an aspect, resuspending the trypsinized cells can comprise PBS and DNase. In an aspect of a disclosed method, fixing the resuspended cells can comprise paraformaldehyde. Methods of fixation and agents for fixation are known to the art.

In an aspect of a disclosed method, during flow cytometry, the cells can be analyzed in two selection gates prior to fluorescence evaluation.

In an aspect, analyzing the cells in two selection gates can comprises (i) identifying cells by evaluating sample forward vs. side scatter area; and (ii) identifying single cells by evaluating cells that maintained a linear correlation of forward scatter height to forward scatter area.

In an aspect, incubating the combined populations of cells can comprise further comprising generating one or more fluorescently-labeled, C-terminally fused connexin constructs. Isolated nucleic acid molecules comprising a nucleic acid sequence encoding a connexin protein are disclosed herein.

In an aspect, a disclosed first fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof. In an aspect, a disclosed second fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof.

In an aspect, a disclosed first connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof. In an aspect, a disclosed second connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof.

In an aspect, a disclosed first connexin protein can comprise the sequence set forth in any one of SEQ ID NO:01-SEQ ID NO:21, or a fragment thereof. In an aspect, a disclosed first connexin protein can comprise the sequence set forth in any sequence provided in Table 5. In an aspect, a disclosed second connexin protein can comprise the sequence set forth in any one of SEQ ID NO:01-SEQ ID NO:21, or a fragment thereof. In an aspect, a disclosed second connexin protein can comprise the sequence set forth in any sequence provided in Table 5.

In an aspect, a disclosed connexin construct can comprise a connexin suspected of having an etiological influence in a disease or disorder. In an aspect, a disclosed connexin construct can comprise a connexin confirmed to have an etiological influence in a disease or disorder.

In an aspect, flow cytometry can generate a fluorescent profile for each cell sample or each population of cells.

In an aspect, a disclosed fluorescence profile can comprise 4 quadrants. In an aspect, disclosed quadrants can comprise Q1, Q2, Q3, and Q4. In an aspect, a disclosed Q1 can represent a population of cells with the first fluorescently-labeled, C-terminally fused connexin construct. In an aspect, a disclosed Q2 can represent a population of cells expressing both the first fluorescently-labeled, C-terminally fused connexin construct and the second fluorescently-labeled C-terminally fused connexin construct. In an aspect, a disclosed Q3 can represent a population of cells with the second fluorescently-labeled C-terminally fused connexin construct. In an aspect, a disclosed Q4 can represent untransfected cells in either the first population of cells or the second populations of cells.

In an aspect, a disclosed method of interrogating the docking interactions between connexins can further comprise establishing a FETCH score.

In an aspect, a disclosed FETCH score can be a positive FETCH score or a negative FETCH score. In an aspect, a disclosed positive FETCH score can indicate a docking event. In an aspect, a disclosed negative FETCH score can indicate a non-docking event. In an aspect, a disclosed negative FETCH score can indicate dysfunctional gap junction intercellular communication. In an aspect, a disclosed FETCH score can comprise the proportion of dual colored-cells compared to fluorescent cells. In an aspect, a disclosed proportion can comprise Q2/(Q1+Q2+Q3).

In an aspect, a disclosed method of interrogating the docking interactions between connexins can comprise quantifying the fluorescence exchange between cells mediated by connexosomes. In an aspect, a disclosed method of interrogating the docking interactions between connexins can comprise confirming the relationship between the fluorescence exchange phenotype and the connexosome formation. In an aspect, confirming the relationship between the fluorescence exchange phenotype and connexosome formation can comprise using fluorescence-activated cell sorting (FACS) to collect cells from Q2 for microscopy analysis.

In an aspect, FACS can confirm the formation of dual-labeled connexosomes.

In an aspect, a disclosed method of interrogating the docking interactions between connexins can identify homotypic docking of hemichannels. In an aspect, a disclosed method of interrogating the docking interactions between connexins can identify homotypic docking of hemichannels. In an aspect, disclosed homotypic docking can comprise docking of two hemichannels having a single connexin isoform. In an aspect, a disclosed method of interrogating the docking interactions between connexins can identify heterotypic docking of hemichannels.

In an aspect, a disclosed heterotypic docking can comprise docking of two hemichannels having a different connexin isoform.

In an aspect, disclosed combinations of hemichannel combinations include the combinations set forth below in Table 2 (supra).

In an aspect, a disclosed method of interrogating the docking interactions between connexins can further comprise performing whole-cell, dual-patch clamp analysis on a disclosed combined population of cells. In an aspect, a disclosed method of interrogating the docking interactions between connexins can identify variation in the expression level of one or more disclosed connexin constructs. In an aspect, a disclosed method of interrogating the docking interactions between connexins can identify variation in trafficking of one or more disclosed connexin constructs. In an aspect, a disclosed method of interrogating the docking interactions between connexins can identify variation in stability of one or more disclosed connexin constructs. In an aspect, a disclosed method of interrogating the docking interactions between connexins can identify variation in the turnover rate of one or more disclosed connexin constructs.

In an aspect, a disclosed method of interrogating the docking interactions between connexins does not quantify hemichannel binding affinity.

In an aspect, a disclosed method of interrogating the docking interactions between connexins can detect dysfunctional gap junction formation and/or communication. In an aspect, a disclosed dysfunctional gap junction formation and/or communication can be due to non-conductive hemichannels. In an aspect, a disclosed dysfunctional gap junction formation and/or communication can be due to non-formation of hemichannels.

In an aspect of a disclosed method of interrogating the docking interactions between connexins, a disclosed FETCH score can reflect variation in (i) the expression level of the connexin constructs, (ii) the trafficking of the connexin constructs, (iii) the stability of the connexin constructs, and/or (iv) the turnover rate of the connexin constructs. In an aspect, a disclosed method of interrogating the docking interactions between connexins can be a downstream indicator of connexin hemichannel docking compatibility.

In an aspect, a disclosed method of interrogating the docking interactions between connexins can be scaled for high-throughput analysis.

In an aspect, a disclosed method of interrogating the docking interactions between connexins can evaluate one or more engineered connexin constructs for one or more new docking features. For example, in an aspect, an engineered connexin construct can comprise a modified CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or any combination thereof.

Disclosed herein is method of interrogating the docking interactions between connexins, the method comprising generating fluorescent connexosomes, wherein generating one or more fluorescent connexosomes comprises transfecting a first population of cells with a first fluorescently-labeled, C-terminally fused connexin construct; transfecting a second population of cells with a second fluorescently-labeled, C-terminally fused connexin construct; incubating the transfected populations of cells; trypsinizing the incubated and transfected population of cells; incubating the trypsinized first population of cells with the trypsinized second population of cells; trypsinizing the incubated combined population of cells, resuspending the trypsinized cells, and fixing the resuspended cells; and performing flow cytometry to track the fluorescent connexosomes by quantifying the fluorescence exchange between cells mediated by connexosomes.

In an aspect, a disclosed method of interrogating the docking interactions between connexins can further comprise treating a subject having a disease or disorder characterized by gap junction dysfunction and/or malfunction. In an aspect, gap junction dysfunction and/or malfunction can be confirmed by a disclosed method.

In an aspect, a disclosed method of interrogating the docking interactions can further comprise evaluating gap junction formation. In an aspect, a disclosed method of interrogating the docking interactions can further comprise high-throughput quantification of gap junction hemichannel docking. In an aspect, a disclosed method of interrogating the docking interactions can further comprise evaluating gap junction formation and high-throughput quantification of gap junction hemichannel docking.

In an aspect, a disclosed method of interrogating the docking interactions between connexins can use a disclosed automated pipeline that can receive and process FETCH data. A disclosed automated pipeline is described infra.

### 3. Methods of High-Throughput Quantification of Gap Junction Hemichannel Docking

Disclosed herein is a method of high throughput quantification of gap junction hemichannel docking, the method comprising performing flow enabled tracking of connexosomes in HEK293T cells (FETCH).

In an aspect, performing FETCH can comprises (i) generating fluorescent connexosomes, and (ii) using flow cytometry to track the fluorescent connexosomes in one or more cell samples.

In an aspect, generating one or more fluorescent connexosomes can comprise transfecting a first population of cells with a first fluorescently-labeled, C-terminally fused connexin construct; and transfecting a second population of cells with a second fluorescently-labeled, C-terminally fused connexin construct. In an aspect, performing FETCH can comprise employing one or more plasmids listed in Table 3.

In an aspect, a disclosed method of high throughput quantification of gap junction hemichannel docking can employ one or more plasmids listed in Table 3. In an aspect, one or more disclosed plasmids can comprise any pair of fluorescent labels or fluorophores that is amendable to analysis via flow cytometry and have excitation and emission spectra that can be isolated or separated from each other, thereby enabling the interrogation of the docking interactions.

In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed method can further comprise incubating the transfected populations of cells. In an aspect of a disclosed method, incubating the transfected populations of cells can comprise about 10 hours to about 24 hours. For example, in an aspect, incubating can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, or about 24 hours. In an aspect, incubating can comprise less than 10 hrs or more than 24 hrs. In an aspect, incubating the transfected populations of cells can comprises about 16 hours to about 18 hours.

In an aspect, a disclosed method can further comprise trypsinizing the incubated and transfected population of cells. Trypsinization, methods of trypsinization, and agents for trypsinizing are known to the art.

In an aspect, a disclosed method can further comprise incubating the trypsinized first population of cells with the trypsinized second population of cells. In an aspect of a disclosed method, incubating the combined populations of cells can comprise about 10 hours to about 30 hours. For example, in an aspect, incubating the combined populations of cells can comprise about 10 hrs, about 11 hrs, about 12 hrs, about 13 hrs, about 14 hrs, about 15 hrs, about 16 hrs, about 17 hrs, about 18 hrs, about 19 hours, about 20 hrs, about 21 hrs, about 22 hrs, about 23 hrs, about 24 hrs, about 25 hrs, about 26 hrs, about 27 hrs, about 28 hrs, about 29 hrs, or about 30 hrs. In an aspect, incubating the combined populations of cells can comprise less than 10 hrs or more than 30 hrs. In an aspect, incubating the combined populations of cells can comprise about 20 hrs. In an aspect of a disclosed method, incubating the combined populations of cells can continue to hyperdensity and/or over-confluency.

In an aspect, a disclosed method can comprise trypsinizing the incubated combined population of cells, resuspending the trypsinized cells, and fixing the resuspended cells. In an aspect, resuspending the trypsinized cells can comprise PBS and DNase. In an aspect of a disclosed method, fixing the resuspended cells can comprise paraformaldehyde. Methods of fixation and agents for fixation are known to the art.

In an aspect of a disclosed method, during flow cytometry, the cells can be analyzed in two selection gates prior to fluorescence evaluation. In an aspect, analyzing the cells in two selection gates can comprises (i) identifying cells by evaluating sample forward vs. side scatter area; and (ii) identifying single cells by evaluating cells that maintained a linear correlation of forward scatter height to forward scatter area.

In an aspect, incubating the combined populations of cells can comprise further comprising generating one or more fluorescently-labeled, C-terminally fused connexin constructs. Isolated nucleic acid molecules comprising a nucleic acid sequence encoding a connexin protein are disclosed herein.

In an aspect, a disclosed first fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof. In an aspect, a disclosed second fluorescently-labeled, C-terminally fused connexin construct can comprise CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or a mutant thereof.

In an aspect, a disclosed first connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof. In an aspect, a disclosed second connexin protein can comprise the sequence set forth in UniProtKB sequence P17302, Q9Y6H8, P35212, P36382, P48165, P57773, Q969M2, P08034, P29033, O75712, Q9NTQ9, O95377, O95452, Q6PEY0, P36383, Q5T442, Q8NFK1, Q9UKL4, Q8N144, Q96KN9, A6NN92, or a fragment thereof.

In an aspect, a disclosed first connexin protein can comprise the sequence set forth in any one of SEQ ID NO:01-SEQ ID NO:21, or a fragment thereof. In an aspect, a disclosed first connexin protein can comprise the sequence set forth in any sequence provided in Table 5. In an aspect, a disclosed second connexin protein can comprise the sequence set forth in any one of SEQ ID NO:01-SEQ ID NO:21, or a fragment thereof. In an aspect, a disclosed second connexin protein can comprise the sequence set forth in any sequence provided in Table 5.

In an aspect, a disclosed connexin construct can comprise a connexin suspected of having an etiological influence in a disease or disorder. In an aspect, a disclosed connexin construct can comprise a connexin confirmed to have an etiological influence in a disease or disorder.

In an aspect, flow cytometry can generate a fluorescent profile for each cell sample or each population of cells. In an aspect, a disclosed fluorescence profile can comprise 4 quadrants. In an aspect, disclosed quadrants can comprise Q1, Q2, Q3, and Q4. In an aspect, a disclosed Q1 can represent a population of cells with the first fluorescently-labeled, C-terminally fused connexin construct. In an aspect, a disclosed Q2 can represent a population of cells expressing both the first fluorescently-labeled, C-terminally fused connexin construct and the second fluorescently-labeled C-terminally fused connexin construct. In an aspect, a disclosed Q3 can represent a population of cells with the second fluorescently-labeled C-terminally fused connexin construct. In an aspect, a disclosed Q4 can represent untransfected cells in either the first population of cells or the second populations of cells.

In an aspect, a disclosed method of high throughput quantification of gap junction hemichannel docking can further comprise establishing a FETCH score. In an aspect, a disclosed FETCH score can be a positive FETCH score or a negative FETCH score. In an aspect, a disclosed positive FETCH score can indicate a docking event. In an aspect, a disclosed negative FETCH score can indicate a non-docking event. In an aspect, a disclosed negative FETCH score can indicate dysfunctional gap junction intercellular communication. In an aspect, a disclosed FETCH score can comprise the proportion of dual colored-cells compared to fluorescent cells.

In an aspect, a disclosed proportion can comprise Q2/(Q1+Q2+Q3).

In an aspect, a disclosed method of high throughput quantification of gap junction hemichannel docking can comprise quantifying the fluorescence exchange between cells mediated by connexosomes.

In an aspect, a disclosed method of high throughput quantification of gap junction hemichannel docking can comprise confirming the relationship between the fluorescence exchange phenotype and the connexosome formation.

In an aspect, confirming the relationship between the fluorescence exchange phenotype and connexosome formation can comprise using fluorescence-activated cell sorting (FACS) to collect cells from Q2 for microscopy analysis. In an aspect, FACS can confirm the formation of dual-labeled connexosomes.

In an aspect, a disclosed method of high throughput quantification of gap junction hemichannel docking can identify homotypic docking of hemichannels. In an aspect, a disclosed method of high throughput quantification of gap junction hemichannel docking can identify homotypic docking of hemichannels. In an aspect, disclosed homotypic docking can comprise docking of two hemichannels having a single connexin isoform. In an aspect, a disclosed method of high throughput quantification of gap junction hemichannel docking can identify heterotypic docking of hemichannels. In an aspect, a disclosed heterotypic docking can comprise docking of two hemichannels having a different connexin isoform.

In an aspect, disclosed combinations of hemichannel combinations include the combinations set forth below in Table 2 (supra).

In an aspect, a disclosed method of high throughput quantification of gap junction hemichannel docking can further comprise performing whole-cell, dual-patch clamp analysis on a disclosed combined population of cells.

In an aspect, a disclosed method of high throughput quantification of gap junction hemichannel docking can identify variation in the expression level of one or more disclosed connexin constructs. In an aspect, a disclosed method of high throughput quantification of gap junction hemichannel docking can identify variation in trafficking of one or more disclosed connexin constructs. In an aspect, a disclosed method of high throughput quantification of gap junction hemichannel docking can identify variation in stability of one or more disclosed connexin constructs. In an aspect, a disclosed method of high throughput quantification of gap junction hemichannel docking can identify variation in the turnover rate of one or more disclosed connexin constructs.

In an aspect, a disclosed method of high throughput quantification of gap junction hemichannel docking does not quantify hemichannel binding affinity.

In an aspect, a disclosed method of high throughput quantification of gap junction hemichannel docking can detect dysfunctional gap junction formation and/or communication. In an aspect, a disclosed dysfunctional gap junction formation and/or communication can be due to non-conductive hemichannels. In an aspect, a disclosed dysfunctional gap junction formation and/or communication can be due to non-formation of hemichannels.

In an aspect of a disclosed method of high throughput quantification of gap junction hemichannel docking, a disclosed FETCH score can reflect variation in (i) the expression level of the connexin constructs, (ii) the trafficking of the connexin constructs, (iii) the stability of the connexin constructs, and/or (iv) the turnover rate of the connexin constructs.

In an aspect, a disclosed method of high throughput quantification of gap junction hemichannel docking can be a downstream indicator of connexin hemichannel docking compatibility.

In an aspect, a disclosed method of high throughput quantification of gap junction hemichannel docking can be scaled for high-throughput analysis.

In an aspect, a disclosed method of high-throughput quantification of gap junction hemichannel docking can evaluate one or more engineered connexin constructs for one or more new docking features. For example, in an aspect, an engineered connexin construct can comprise a modified CX23, CX25, CX26, CX30, CX30.2/CX31.3, CX30.3, CX31, CX31.1, CX31.9/CX30.2, CX32, CX36, CX37, CX40, CX40.1, CX43, CX45, CX46, CX47, CX50, CX59, CX62, or any combination thereof.

Disclosed herein is a method of high-throughput quantification of gap junction hemichannel docking, the method comprising generating fluorescent connexosomes, wherein generating one or more fluorescent connexosomes comprises transfecting a first population of cells with a first fluorescently-labeled, C-terminally fused connexin construct; transfecting a second population of cells with a second fluorescently-labeled, C-terminally fused connexin construct; incubating the transfected populations of cells; trypsinizing the incubated and transfected population of cells; incubating the trypsinized first population of cells with the trypsinized second population of cells; trypsinizing the incubated combined population of cells, resuspending the trypsinized cells, and fixing the resuspended cells; and performing flow cytometry to track the fluorescent connexosomes by quantifying the fluorescence exchange between cells mediated by connexosomes.

In an aspect, a disclosed method of high-throughput quantification of gap junction hemichannel docking can further comprise treating a subject having a disease or disorder characterized by gap junction dysfunction and/or malfunction. In an aspect, gap junction dysfunction and/or malfunction can be confirmed by a disclosed method.

In an aspect, a disclosed method of high-throughput quantification of gap junction hemichannel docking can further comprise evaluating gap junction formation. In an aspect, a disclosed method of high-throughput quantification of gap junction hemichannel docking can further comprise interrogating the docking interactions between connexins. In an aspect, a disclosed method of high-throughput quantification of gap junction hemichannel docking can further comprise evaluating gap junction formation and interrogating the docking interactions between connexins.

In an aspect, a disclosed method of evaluating gap junction formation can use a disclosed automated pipeline that can receive and process FETCH data. A disclosed automated pipeline is described infra.

### EXAMPLES

Here, a new method called FETCH was developed. FETCH is the first of its kind in specifically interrogating the docking interactions of different Cx isoforms and mutant proteins. FETCH makes strides over traditional methods by enabling the direct, high-throughput, low-skill, and fast analyses, which ultimately allows for the dissection and manipulation of complex interactions throughout the body.

As described herein, FETCH (flow enabled tracking of connexosomes in HEK cells) is a method that utilizes the generation of annular gap junctions (connexosomes) as downstream indicators of connexin hemichannel docking compatibility. First, the data show that fluorescent connexosomes create a cellular phenotype that is detectable by flow cytometry. Second, the data show that FETCH identifies homotypic and heterotypic docking of many single isoform connexin hemichannels. Third, the data show that FETCH captures the impact of disease-relevant connexin protein mutations on gap junction formation. Collectively, the data demonstrate the development of a new flow cytometry-based method that is amenable to the high-throughput classification of gap junction hemichannel docking.

The Examples that follow are illustrative of specific aspects of the invention, and various uses thereof. They set forth for explanatory purposes only and are not to be taken as limiting the invention.

### A. MATERIALS AND METHODS EMPLOYED IN SPECIFIC EXAMPLES

### 1. Construct Cloning and Preparation

Connexin gene information was procured from the National Center for Biotechnology Information (NCBI, ncbi.nlm.nih.gov) and the Ensembl genome browser (ensembl.org). The human codon-optimized genes were ordered from Integrated DNA Technology (IDT, idtdna.com). Genes were subcloned into Emerald-N1 (addgene:53976), piRFP670-N1 (addgene: 45457) and Apple-N1 (addgene: 54567) vectors using In-Fusion cloning (takarabio.com), resulting in connexin fluorescent fusion proteins, specifically with the fluorescent proteins being adjoined to the connexin carboxy-terminus. Previous studies have indicated that carboxy-terminal tagging of connexin proteins is less likely to cause functional perturbations than amino-terminal tagging (Contreras J E, et al. (2003) Proc Natl Acad Sci USA. 100:11388-11393).

Mutant constructs were generated using overlapping primers within standard Phusion polymerase, PCR amplifications to facilitate site-directed mutagenesis.

TABLE 4
Primers Listing in Sequence Listing.

| SEQ ID NO: | Primer Description |
|---|---|
| 87 | Cx43-S18P-top primer |
| 88 | Cx43-S18P-bot primer |
| 89 | Cx43-A40V-top primer |
| 90 | Cx43-A40V-bot primer |
| 91 | Cx43-N55D-top primer |
| 92 | Cx43-N55D-bot primer |
| 93 | Cx43-Q58H-top primer |
| 94 | Cx43-Q58H-bot primer |
| 95 | Cx43-P59A-top primer |
| 96 | Cx43-P59A-bot primer |
| 97 | Cx43-S69Y-top primer |
| 98 | Cx43-S69Y-bot primer |
| 99 | Cx43-F70A-top primer |
| 100 | Cx43-F70A-bot primer |
| 101 | Cx43-I130T-top primer |
| 102 | Cx43-I130T-bot primer |
| 103 | Cx43-F179A-top primer |
| 104 | Cx43-F179A-bot primer |
| 105 | Cx43-S182A-top primer |
| 106 | Cx43-S182A-bot primer |
| 107 | Cx43-V184A-top primer |
| 108 | Cx43-V184A-bot primer |
| 109 | Cx43-D190A-top primer |
| 110 | Cx43-D190A-bot primer |
| 111 | Cx43-H194A-top primer |
| 112 | Cx43-H194A-bot primer |
| 113 | Cx43-R202H-top primer |
| 114 | Cx43-R202H-bot primer |
| 115 | Cx43-V216L-top primer |
| 116 | Cx43-V216L-bot primer |

### 2. Cell Culture

HEK 293FT cells were purchased from Thermo Fisher Scientific (cat #R70007) and were maintained according to manufacturer's instructions. Cultures were grown in 10 cm tissue culture treated dishes in high-glucose DMEM (Sigma Aldrich, D5796) supplemented with 6 mM L-glutamine, 0.1 mM MEM non-essential amino acids, and 1 mM MEM sodium pyruvate in a 5% CO₂, 37° C. incubator. Cells were passaged via trypsinization every 2-3 days or until 60-80% confluency was reached.

### 3. Transient Transfection:

HEK 293FT cells were plated in 10 μg/mL fibronectin coated multi-well dishes to achieve ˜75% confluency after overnight incubation. For transfection, DNA was combined with polyethylenimine (PEI) diluted in Opti-MEM in a 1:3 ratio (μg of DNA:μL of PEI) and incubated at room temperature for 10 min. Following incubation, PEI-DNA complexes were added dropwise to wells of plated cells. Treated cells were then incubated at 37° C. for 16-18 hrs, followed by media change. Expression of the connexin-FP constructs was evaluated at 24 hrs and 48 hrs post-transfection via widefield or confocal microscopy and western blotting.

### 4. Confocal Imaging Analysis

For imaging, HEK 293FT were plated in 10 μg/mL fibronectin coated 35 mm, glass-bottom Mattek dishes (cat #P35GC-1.5-14-C). Cells were transiently transfected using PEI as described above and imaged at ˜48 hrs post-transfection. Images were acquired on a Leica SP5 laser point scanning inverted confocal microscope using Argon/2, HeNe 594 nm and HeNe633 nm lasers, conventional fluorescence filters and a 63X, HCX PL APO W Corr CS, NA: 1.2, Water, DIC, WD: 0.22 mm, objective. Images were taken with 1024×1024 pixel resolution at 200 Hz frame rate.

### 5. Cell Lysate Preparation and Western Blotting

To extract cell lysates, plated cells were washed twice with room temperature PBS, trypsinized, collected, and centrifuged at 2,000×g for 5 minutes. Pellets were washed twice with PBS followed by centrifugation and aspiration. Pellets were lysed by the addition of 10 mM Tris, pH 8.0, 1 mM EDTA, 1% SDS, and 1× protease and phosphatase inhibitor cocktail (Thermo) and sonicated. Lysates were quantified via detergent compatible Bradford assay (ThermoFisher). For western blot, samples were separated using gel electrophoresis on 4-20% SDS-Page gels. Separated proteins were transferred to PVDF membrane in transfer buffer (25 mM Tris, 190 mM glycine, 20% methanol, final pH 8.3), for 1 hr at 110 V. Subsequently, membranes were blocked in 5% milk for 1 hr, followed by 1 hr incubation with primary antibody (1:1000) in 5% milk with shaking. Blots were then washed three times with TBST (20 mM Tris, pH 7.5, 150 mM NaCl and 0.1% Tween 20) for 5 minutes and incubated with Horse radish peroxidase conjugated secondary antibody (1:5000), followed by three washes with TBST for 5 minutes. Blots were developed using Optiblot ECL kit (Abcam) and visualized via Azure c300 phosphorimager. To re-stain individual blots for different proteins, blots were stripped with mild stripping buffer (200 mM glycine, 0.1% w/v SDS, 1% Tween 200), 2×10-minute washes with shaking, followed by 2×10-minute PBS washes with shaking and 2×5-minute TBST washes with shaking. After stripping, blots were blocked in 5% milk for 1 hr in preparation for antibody staining as previously described.

### 6. Flow Enabled Tracking of Connexosomes in HEK 293FT Cells (FETCH)

FETCH analysis is fundamentally a two-component system. To complete FETCH analysis, replica multi-well plates were transfected with either of the two components being evaluated. The media of transfected wells was changed 16-18 hrs post-transfection and cells were trypsinized and experimental counterparts were combined at ˜20 hrs post-transfection. The entirety of combined samples were then plated onto new, 10 μg/mL Fibronectin coated wells of the same size, resulting in hyperdensity and over confluency. Following co-plating, samples were incubated for ˜20 hr-24 hrs, at which point samples were trypsinized, resuspended in phosphate buffered saline with 10 U/mL DNAse and fixed with paraformaldehyde (f/c of 1.5%). Co-plated samples in 96 well plates were resuspended to a final volume of ˜150 μL, samples from 24-well plates were resuspended to a final volume of ˜600 μL.

Flow cytometry data was collected on either a BD FACSCanto II (2-color experiments and high-throughput 96-well plates; 488 nm and 633 nm lasers), which utilizes the BD FACSDiVa software. Samples were analyzed in two selection gates prior to their fluorescence evaluation. First, presumable HEK cells were identified by evaluating sample forward vs side scatter area. Next, single cells were selected by evaluating cells that maintained a linear correlation of forward scatter height to forward scatter area. Finally, the fluorescence profiles of each sample were generated.

### 7. Fetch Automated Gating Pipeline

Each FETCH experiment produces “*.fcs” files that contain all the channel data for fluorescence in the sample. The automated pipeline loads these files, extracts FSC-A, SSC-A, and FSC-H. Depending on the machine used, green channel was loaded as 1-A or as FITC-A. For the red channel, both APC-A (RFP670) with PE-A (mApple) can be used or 5-A(RFP670) can be used. Next, the code produces two matrices containing SSC-A with FSC-A, and FSC-A with FSC-H, respectively.

The first gate is drawn on the FSC-A vs SSC-A axes to exclude cellular debris which clusters in the lower left corner and the cells that are saturating the laser (at the max of both axes). On an FSC-A vs SSC-A plot, the cellular debris usually is smoothly transitioning into the population of intact cells, therefore a Gaussian kernel density estimator with the estimator bandwidth selection defined by the Scott's Rule is used to draw contours around the data in SSC-A vs FSC-A matrix. Next, a set of heuristics was used to determine which of the contour lines should be used to define the first gate. Specifically, cellular debris usually clusters below 25000 on both axes, so any contour that includes values at or below is excluded. Similarly, any contour within a 1000 of the maximum value of each axis is also excluded. Of the remaining contours, the largest one is chosen, and an oval equation is fitted to the points defining that contour to attenuate occasional protrusions of the contour that tap into cellular debris population in rare cases. The fitted oval becomes the first gate.

For all the elements inside of the first gate, a second gate was drawn in the axis of FSC-A and FSC-H to exclude non-single cells. For the second gate, a line was fit to all the points. Next, for each point, a norm to the fit line was found and a standard deviation of all such norms was found. Using this standard deviation, a second gate 4 standard deviations away from the fitted line was defined on both sides and excluded all the points outside of this gate.

Upon applying the first two gates, the data was finally plotted with the red fluorophore on the y axis and the green fluorophore on the x axis. If a sample contained more than two fluorophores, then the last gate was drawn for each possible combination. Since some readings are below zero due to fluorescence compensation, all the data points were shifted by the smallest value along both axes and then a natural log of fluorescence levels was taken. To achieve the optimal bandwidth for the kernel density estimation, a cross validation grid search algorithm was run on the points in the log space. A gaussian kernel density with the bandwidth estimated to obtain density contours was fit.

For properly expressing samples, the expectations were as follows: (1) a large population of untransfected cells in the bottom left quadrant of the plot, (2) a population of cells strongly-expressing red fluorophore along the y axis, and (3) a population of cells strongly expressing green fluorophore along the x axis. Autofluorescence was expected to not exceed 500 on either axis, so the untransfected population was defined to be below this value along both axes. To draw a tighter bound of the untransfected population, the first contour whose mean kde value is at or above the 60th quantile (identified as a generalizable heuristic value) of the distribution of kde values within the largest contour which is at or below the autofluorescence cutoff was chosen. The top-most point of the tight contour defined the horizontal gate and the right-most point—the vertical gate, separating the plot into four quadrants.

The upper left quadrant Q1 corresponds to the cells expressing just the red fluorophore, the upper right quadrant Q2 represents dual-colored cells, the lower right quadrant Q3 shows the cells expressing just the green fluorophore, and the lower left quadrant Q4 represents untransfected population. FETCH score was defined as the proportion of dual-colored cells to all transfected cells: Q2/(Q1+Q2+Q3).

Expecting approximately equal expression levels of each fluorophore, if the number of cells in Q1 is two or more times larger/smaller than the number of cells in Q3, then the FETCH score was classified as “dubious” and marked accordingly in the output table. The “dubious” label was also given to samples that have less than 500 cells total after the application of the second gate and to the samples that failed at any of the steps in the pipeline (usually due to poor expression or the absence of cells in the sample).

8. Fluorescence Recovery after Photobleaching (FRAP) Analysis:

For FRAP analysis, HEK 293FT cells were plated into 10 μg/mL fibronectin coated mattek glass-bottom dishes (MatTek Corporation, cat #: P35G-1.5-14-C) to achieve ˜75% confluency after overnight incubation. Dishes were then transfected with connexin constructs and allowed to incubate overnight. After 16 hrs-18 hrs, transfection media was replaced with fresh media and FRAP was initiated at ˜24 hrs post-transfection. For FRAP, 1 μg/mL Calcein-AM (Enzo Life Sciences, cat #: 52002) was added to dishes, incubated at 39° C., 5% CO₂ for 30 minutes and then aspirated. Dishes were rinsed 3 times with phosphate buffered saline and then imaged in Tyrode's Solution (Alfa Aesar, cat #:J67593-AP).

Imaging was completed on a Leica SP5 confocal microscope using the LAS Software FRAP Wizard. The experimental program centered on imaging with a 63× water immersion objective, 512×512 Hz and staging pre-bleach (10 frames, 1.29 s/f), bleach (30 frames, 1.29 s/f) and recovery (24 frames, 10 s/f) imaging intervals. For bleaching conditions, the Argon laser was set to 20% power and full power was used to bleach a selected cell ROI. Cells expressing the connexin construct of interest were selected for bleaching if they were surrounded by at least 3 cells making clear contacts and also clearly expressing the exogenous construct as determined by fluorescence.

FRAP images were analyzed using a custom written FIJI plugin and python script. Using a semi-automatic FIJI plugin, 2 ROIs were drawn: one around the bleached cell, and the other—around a cell that did not get bleached. Raw fluorescence was extracted from those and saved into a file for each frame in a stack. Files were then processed using the custom python script, first normalizing the fluorescence of the bleached cell to the fluorescence of the unbleached cell for each frame, and then calculating the percent fluorescence of the baseline by dividing the resultant value by the mean corrected value of the pre-bleaching frames. Since many mutants exhibited low to no fluorescence recovery, it was impossible to fit a reasonable exponential curve to them, impeding the calculation of time constant and similar fitted metrics. Instead, the percent of the baseline value at the end of an imaging experiment was used for statistical analysis.

### B. SPECIFIC EXAMPLES

### Example 1

Dual Fluorescently-Labeled Connexosomes were Detected by Flow Cytometry

Connexosomes are cytoplasmic, double-bilayer vesicular structures comprised of tens to thousands of fully-docked gap junction channels that have been internalized from gap junction plaques (Jordan K, et al. (2001) J Cell Sci. 114:763-773). As connexosomes are the direct products of gap junction turnover, it was reasoned that they could be used as indicators of gap junction docking and, by extension, as indicators of the compatibility of connexin hemichannels.

Connexin43 (Cx43) is one of the best characterized connexin isoforms, and C-terminally fused fluorescent Cx43 constructs were among the first used to observe and characterize the dynamic features of connexosomes including internalization rate, vesicular sizes, and cellular fates (Gaietta G, et al. (2002) Science. 296:503-507; Jordan K, et al. (2001) J Cell Sci. 114:763-773; Piehl M, et al. (2007) Mol Biol Cell. 18:337-347). Thus, two-color, fluorescent connexosomes of Cx43 were generated and the feasibility of high-throughput connexosome identification in cells was determined.

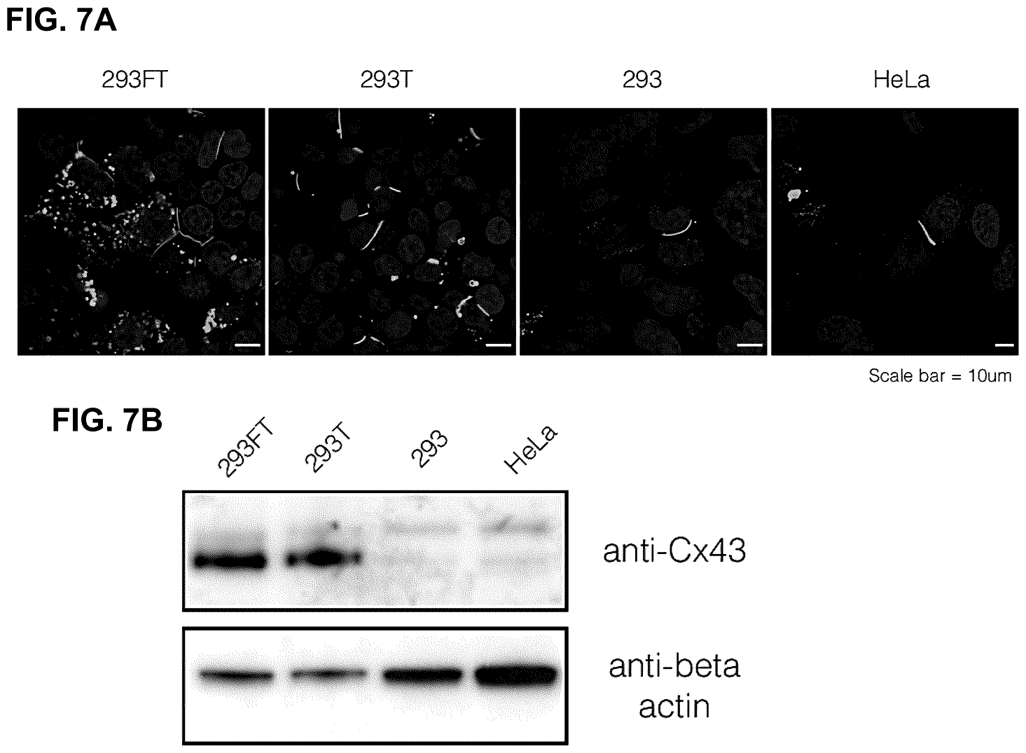

Previous studies have evaluated endogenous connexosomes in native tissues and cells, such as membrana granulosa cells of the rabbit ovarian follicle (Espey L L, et al. (1972) Biol Reprod. 6:168-175; Larsen W J. (1978) Tissue Cell. 10:585-598; Larsen W J, et al. (1981) Biol Reprod. 25:1119-1134; Albertini D F, et al. (1975) Tissue Cell. 7:389-405), guinea pig epithelia (Archard H, et al. (1979) J Oral Path. 8(4):187-197), or the adrenocortical tumor cell line (SW-13) (Murray S A, et al. (1981) Cancer Res. 41:4063-4074; Murray S A, et al. (2004) Endocr Res. 30:647-654; Bell C L, et al. (2019a) Int J Mol Sci. 20(1):44), or as products of exogenous construct expression in HeLa cells (Fong J T, et al. (2012) Autophagy. 8:794-811; Gaietta G, et al. (2002) Science. 296:503-507; Piehl M, et al. (2007) Mol Biol Cell. 18:337-347) or normal rat kidney (NRK) cells (Jordan K, et al. (2001) J Cell Sci. 114:763-773). The HEK 293FT cell line (ThermoFisher) was selected for these studies as HEK 293T cells had substantially higher protein expression and larger gap junction plaque sizes when compared to HeLa and other HEK derivative cell lines. (FIG. 7A-FIG. 7B).

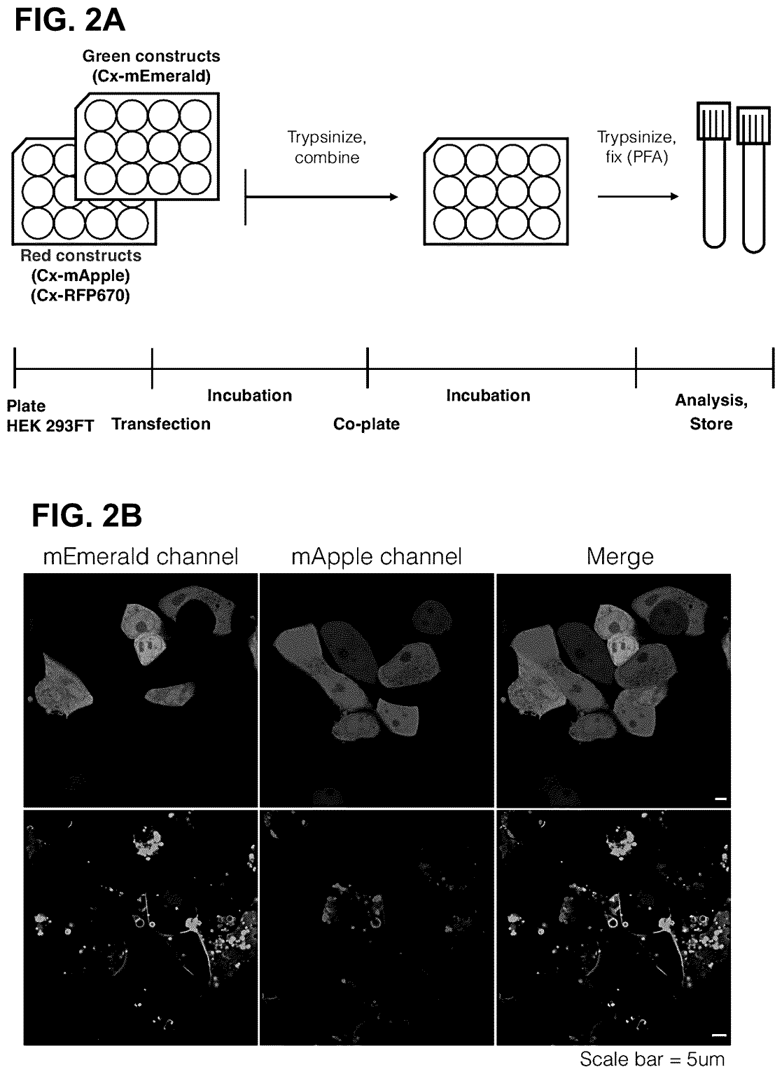

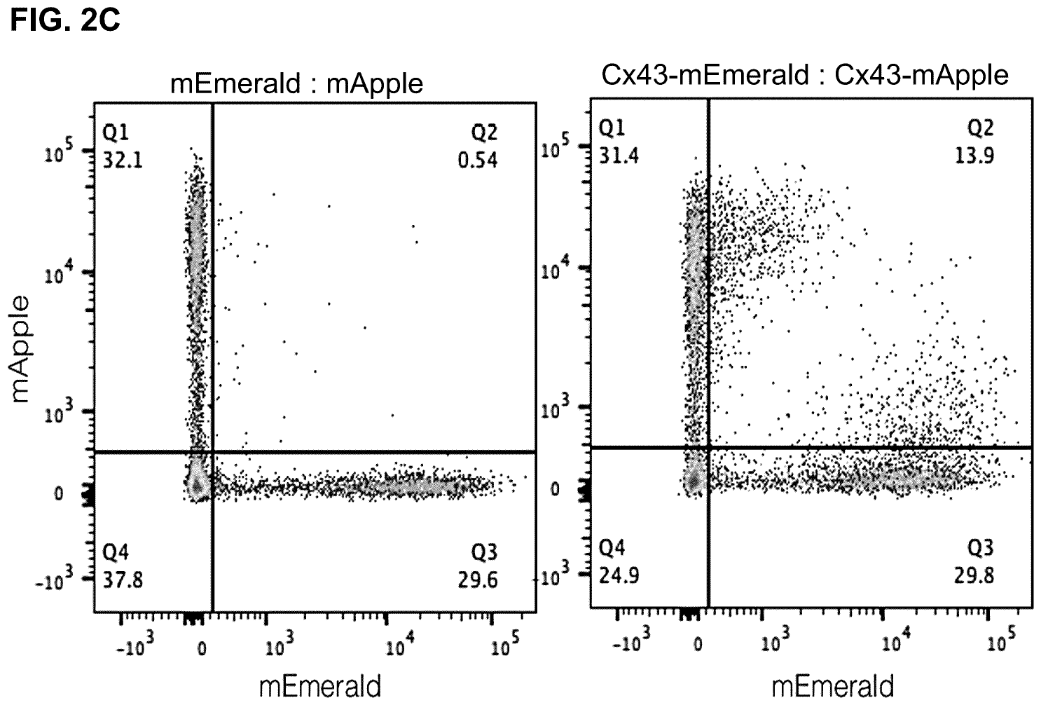

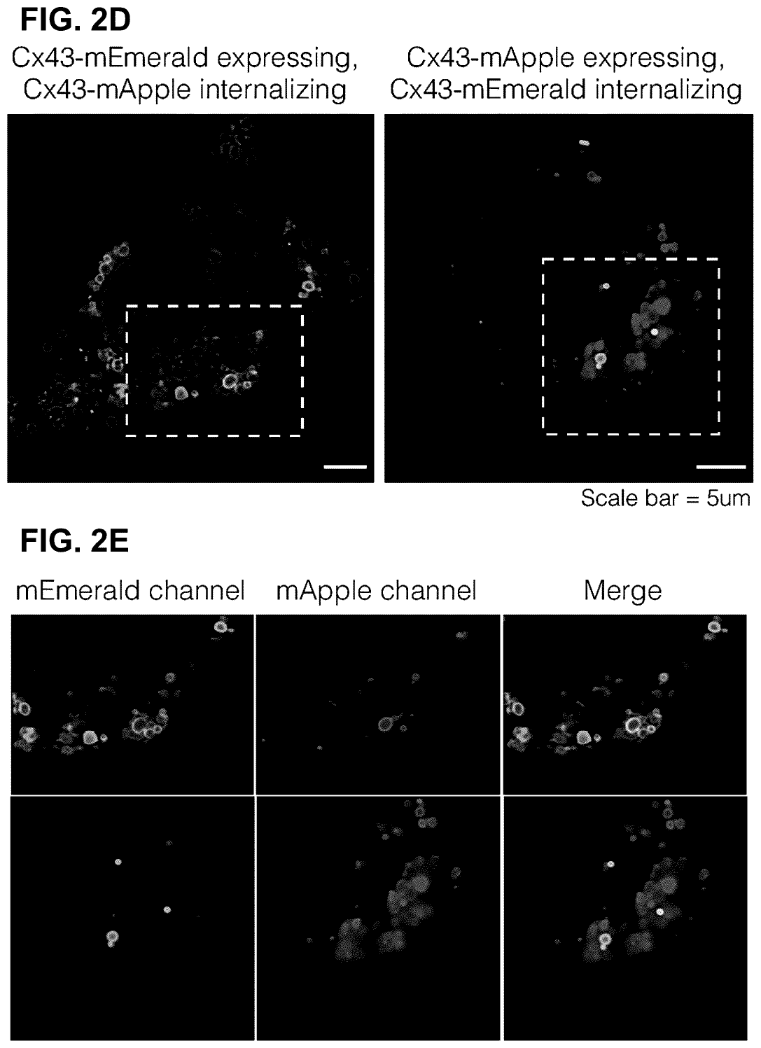

To generate two-color connexosomes and characterize them using microscopy, separate populations of cells were transfected with one of two fluorescently labeled, C-terminally fused Cx43 constructs (i.e., either Cx43-mEmerald or Cx43-mApple). Following an initial incubation, transfected cells were trypsinized, co-plated, and incubated together for-18 hours prior to analysis (FIG. 2A). Transfection of HEK 293FTs with fluorescently-tagged Cx43 constructs resulted in the generation of connexosomes. Specifically, when co-plated Cx43-mEmerald and Cx43-mApple expressing cells were examined, junctional plaques with dual-fluorescent co-localized invaginations were observed. This is indicative of active connexosome formation and the dual-fluorescent vesicular structures were consistent with connexosomes (FIG. 2B) (Piehl M, et al. (2007) Mol Biol Cell. 18:337-347). In contrast, co-plated cells expressing cytoplasmic fluorescent proteins did not generate fluorescently labeled vesicles or plaque structures (FIG. 2B).

Flow cytometry generates multi-wavelength fluorescence profiles of individual cells within a given population. Thus, it was reasoned that this analysis could be used to quantify fluorescence exchange between cells mediated by connexosomes in a high-throughput fashion. This novel approach is called flow enabled tracking of connexosomes in HEK 293FT cells (FETCH). For FETCH analyses, two-color connexosomes were generated as described previously. But instead of imaging, cells were trypsinized and fixed in suspension for evaluation by flow cytometry. As a control, samples of co-plated cells expressing cytoplasmic mEmerald and mApple were analyzed as well.

Upon generation of cellular fluorescence profiles by flow cytometry, a significant phenotypic difference between samples that generated connexosomes (i.e., Cx43-fluorescent protein) and those that did not (i.e., cytoplasmic fluorescent protein) emerged. For both samples, subpopulations of untransfected cells, mApple-labeled cells, and mEmerald-labeled cells were clearly identifiable (FIG. 2C—Q4 (bottom left), Q1 (upper left), and Q3 (bottom right), respectively). But for the Cx43-mEmerald/Cx43-mApple sample, a significant proportion of cells exhibited both green and red fluorescence (FIG. 2C, right panel, Q2 (upper right)). This unique double-positive or dual-fluorescent phenotype was characterized by symmetrical population shifts from the single-expressing quadrants (meaning, specifically, cells highly expressing Cx43-mEmerald gained Cx43-mApple fluorescence and cells highly expressing Cx43-mApple gained Cx43-mEmerald fluorescence). Importantly, acquisition of the secondary fluorescent construct was commensurate with but not equal to the primary fluorescent construct. These observations were consistent with fluorescence exchange mediated by connexosomes.

To confirm the relationship between the fluorescence exchange phenotype of the Cx43-mEmerald/Cx43-mApple sample and connexosome formation, fluorescence-activated cell sorting (FACS) was used to collect cells from each subpopulation of the +/+ quadrant for microscopy analysis. Confocal imaging of the FACS sorted double-positive cells revealed dual-labeled vesicles consistent with connexosomes (FIG. 2D). Moreover, the configuration of fluorescent labels of the connexosomes was such that the expressed, primary construct labeled the outer surface of the connexosome and the acquired, internalized construct was on the interior. This pattern was observed for both Cx43-mEmerald and Cx43-mApple expressing cells. Though post-sorted cells showed significant amounts of vesicular structures that were single-labeled (likely representing transport vesicles and internalized hemi-plaques), no single-labeled vesicles of secondary fluorescent constructs were observed. This finding indicates that the dual-labeled vesicles were connexosomes and that connexosomes generated the observed fluorescence phenotype. Overall, these results demonstrated that FETCH could be used to capture the fluorescence exchange between cells, mediated by connexosomes, and indicative of gap junction docking.

### Example 2

### FETCH Evaluated Docking Interactions of Numerous Connexin Protein Isoforms

As the docking principles that generate annular gap junctions are common to all connexin proteins, it was reasoned that connexin isoforms with documented participation in homotypic gap junctions would be amenable to FETCH analysis. To standardize the quantification of fluorescence exchange, a metric was established. This metric—known as the FETCH score—represents the proportion of the +/+ quadrant cells, adjusted for total fluorescent events, minimizing the influence of transfection efficiency variability on results. Known positive and negative FETCH score distributions representative of non-docking combinations were generated for statistical comparison.

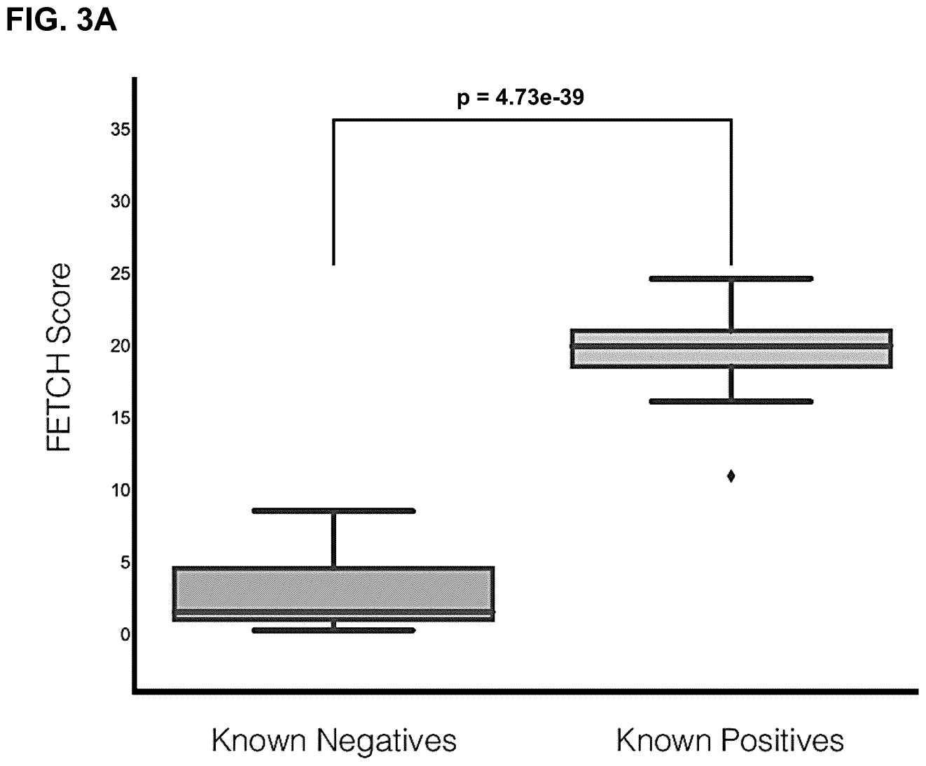

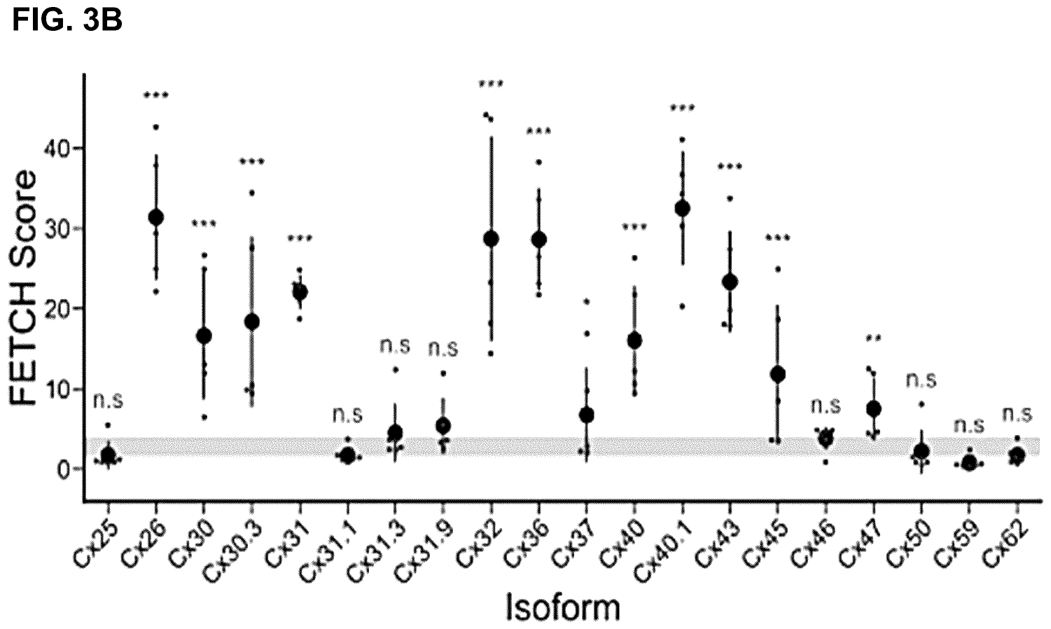

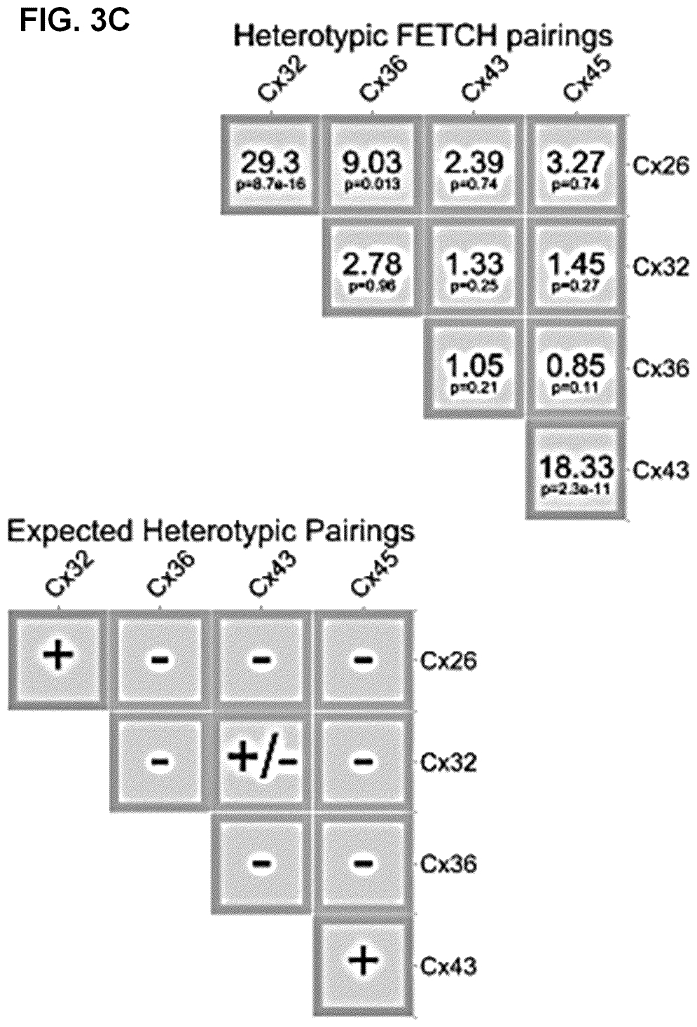

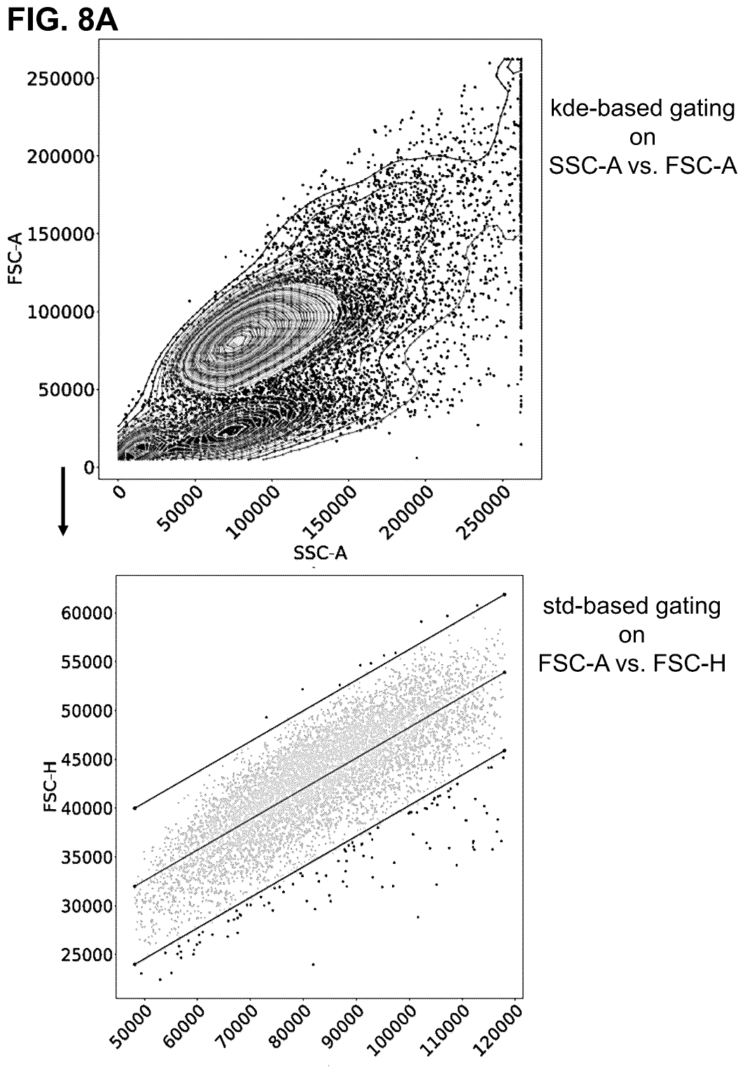

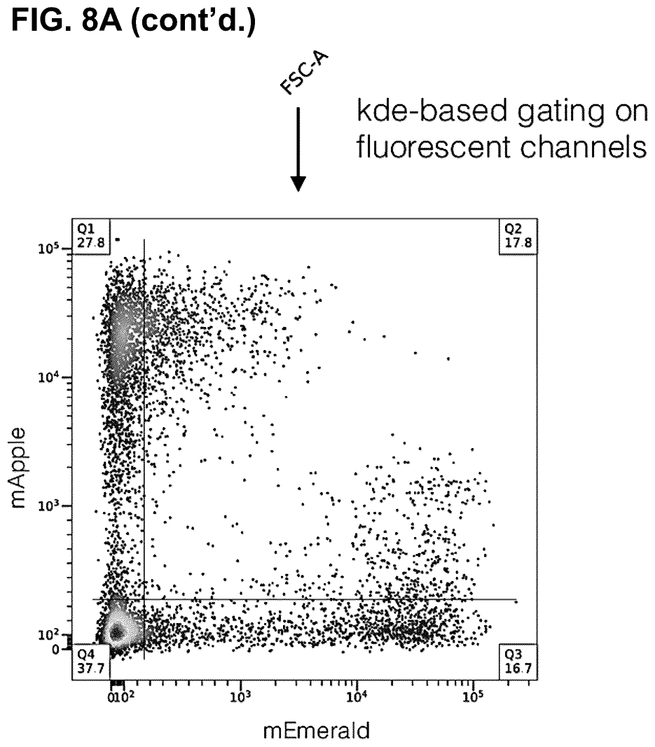

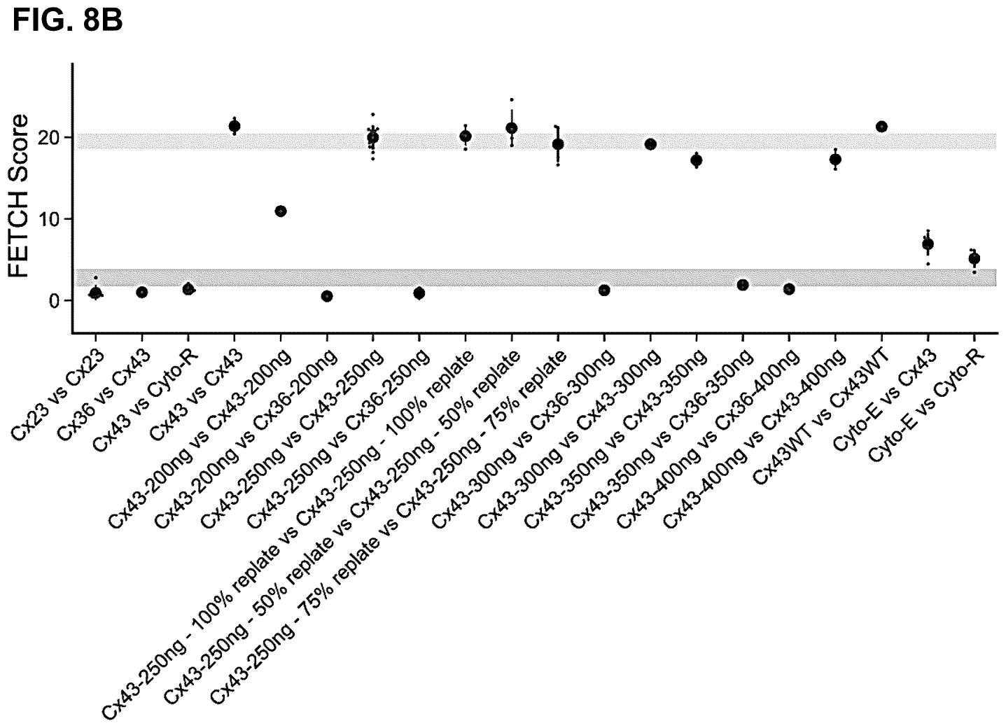

To generate a positive distribution, samples of homotypic pairings of Cx43-FPs with varied DNA transfection quantities and co-plated population densities to capture physical condition variabilities (i.e., small volume pipetting error during transfection and co-plating) were evaluated. Additionally, homotypic pairings of Connexin36 (Cx36)-FPs, a neuronal connexin isoform that forms homotypic gap junctions (Deans M R, et al. (2002) Neuron. 36:703-712; Rash J E, et al. (2001) Cell Commun Adhes. 8:315-320), were evaluated. (FIG. 3A, FIG. 8B).

The negative distribution was comprised of samples of co-plated cytoplasmic fluorescent protein (FP) expressing cells (i.e., mEmerald with mRFP670) and samples of co-plated cells expressing Cx43-FP and cytoplasmic FP (i.e., Cx43-GFP with cytoplasmic RFP).

Additionally, samples of non-docking combinations of different Cx isoforms were evaluated. Specifically, co-plated cells expressing Cx43-FP with cells expressing Cx36-FP (in heterotypic combination) and cells expressing Cx23-FP (homotypically) (FIG. 3A, FIG. 8B). Though Cx36 and Cx43 individually form homotypic gap junctions in the mammalian nervous system, they have not been reported to form heterotypic channels and Cx23 fails to form docked gap junctions due to a missing EL disulfide bond (Sonntag S, et al. (2009) Eur J Cell Biol. 88:65-77).

To evaluate the docking ability of other Cx family members using FETCH, mEmerald and RFP670 C-terminal fusion constructs of the 18 remaining connexin isoforms were generated and subjected to a complete homotypic FETCH analyses. FETCH scores for many Cx isoforms were significantly higher than the distribution for negative docking conditions, indicating that most isoforms are amenable to FETCH analysis. The observed variability in these FETCH scores likely reflected isoform specific characteristics (e.g., expression level, trafficking, stabilization, and turnover rate).

Using an independent two-sample t-test with FDR correction, it was determined that Cx25, Cx31.1, Cx31.3, Cx31.9, Cx46, Cx50, Cx59, and Cx62 failed to generate a significant homotypic FETCH score consistent with docking (FIG. 3B).

Next, to validate the specificity of FETCH in capturing compatible docking interactions, select Cx isoforms were tested for heterotypic interactions. Connexin isoforms are classified by sequence homology into 5 groups (alpha (A), beta (B), gamma (C), delta (D), epsilon (E)) (Beyer E C, et al. (2009). The family of connexin genes. In Connexins (pp. 3-26). Humana Press). Some connexin isoforms can form heterotypic (two-type) channels, frequently with other isoforms within the same homology class, and with some exceptions, isoforms in different classes are not as widely compatible for heterotypic docking (Harris AL (2001) Q Rev Biophys. 34(3):325; Koval M, et al. (2014) Febs Letters. 588:1193-1204). Thus, a heterotypic FETCH evaluation was performed with well-characterized representatives of each major class of connexin. Specifically, in addition to Cx43, also chosen were Cx26, Cx32, Cx45, and Cx36 as representatives to evaluate heterotypic, combinatorial pairings. Importantly, each of these representatives exhibited significantly positive FETCH scores in homotypic evaluations.

Both Cx26 and Cx32 reportedly failed to form functional heterotypic gap junctions with Cx43 in Xenopus laevis oocyte experiments (White T, et al. (1995) Mol Biol Cell. 6(4):459-470). On the other hand, Cx45 is frequently co-expressed in the same tissues as Cx43 and has been formed Cx43/Cx45 heterotypic channels (Moreno A P, et al. (1995) Prog Cell Res. 4:405-408).

When the completed heterotypic FETCH on all combinations of the subset of isoforms was completed, the results showed that the most cross-class heterotypic connexin combinations yielded negative FETCH scores, reflecting docking incompatibility (FIG. 3C). Using independent two-sample t-tests with FDR correction, the FETCH scores were used to determine statistical significance. Consistent with expectations, FETCH analysis revealed that Cx26, Cx32, and Cx36 failed to dock with Cx43, while Cx45 showed a positive FETCH score when paired with Cx43 (n=7, z=18.33, p=2.3×10⁻¹¹) (FIG. 3C).

In addition to the Cx43/Cx45 pairing, a positive FETCH score was observed when pairing Cx26 with Cx32 (n=6, z=29.30, p=8.7×10⁻¹⁶), which belong to the same class, have homologous extracellular loop docking motifs, and have also been reported to form functional heterotypic gap junctions (Barrio L C, et al. Proc Natl Acad Sci USA. 88:8410-8414; Gong X Q, et al. (2013) J Cell Sci. 126:3113-3120) (FIG. 3C).

Interestingly, a positive FETCH score was observed when pairing Cx26 and Cx36 (n=5, z=9.03, p=0.013). This is an unreported pairing that can represent a newly discovered functional compatibility. These results demonstrate that the FETCH method captured physiological gap junction formation and can be applied to broader evaluations of other connexin isoforms in diverse and uncharacterized combinations.

### Example 3

### FETCH Captured Functional Docking of Disease Relevant Connexin Mutant Proteins

Cx43 is the most ubiquitously expressed connexin in the body with critical, tissue-specific functions in nearly 40 tissues and organs including the heart, brain, liver and skin. Unsurprisingly, mutations in the GJA1 gene, which encodes Cx43, often result in numerous, systemic physiological perturbations. The majority of Cx43 disease-associated mutations result in the complex developmental disorder, Oculodentodigital dysplasia (ODDD) (Paznekas W A, et al. (2003) Am J Hum Genet. 72:408-418; Laird D W (2014) Febs Letters. 588:1339-1348), which is characterized by congenital craniofacial, limb and digital deformities, teeth and eye anomalies, and neurological dysfunction (De Bock M, et al. (2013) Front Pharmacol. 4:120).

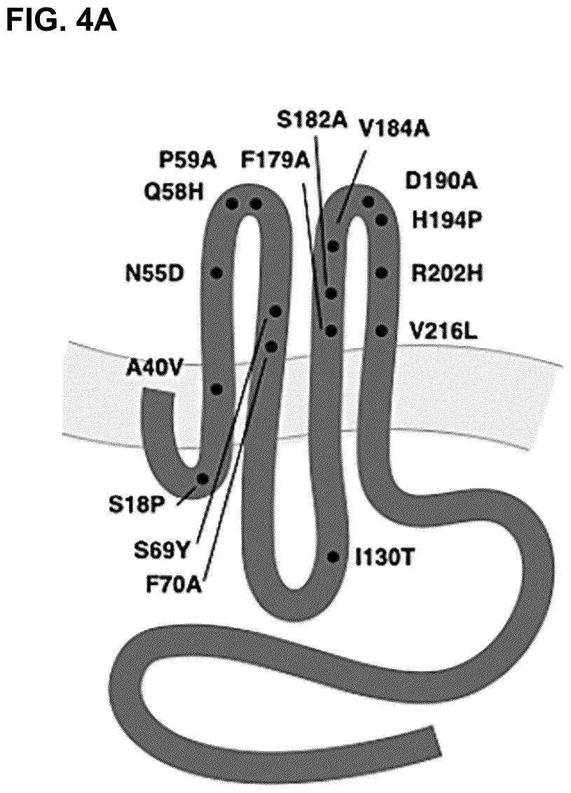

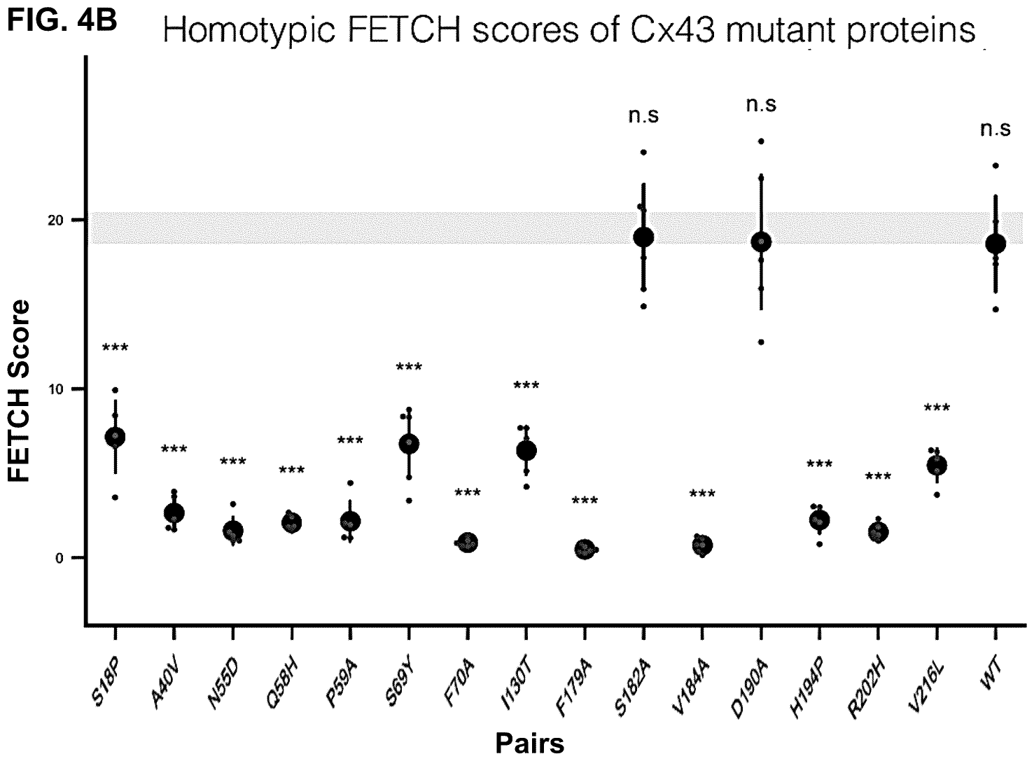

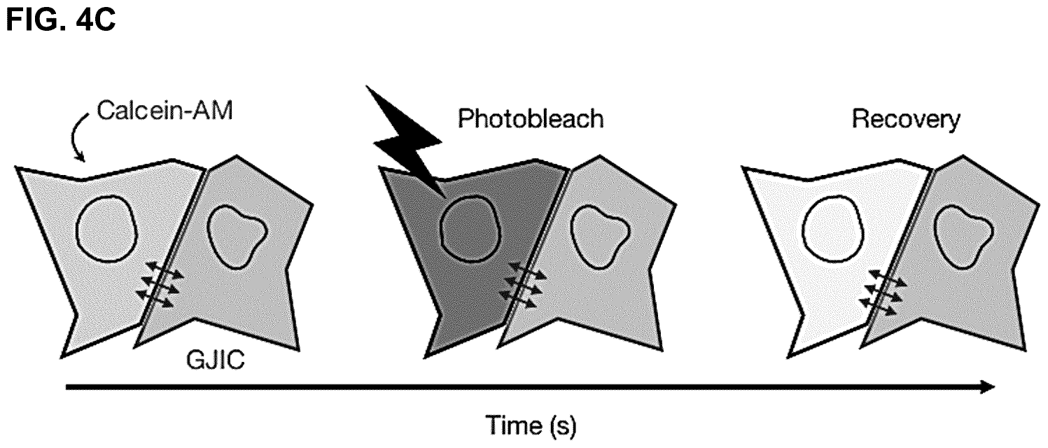

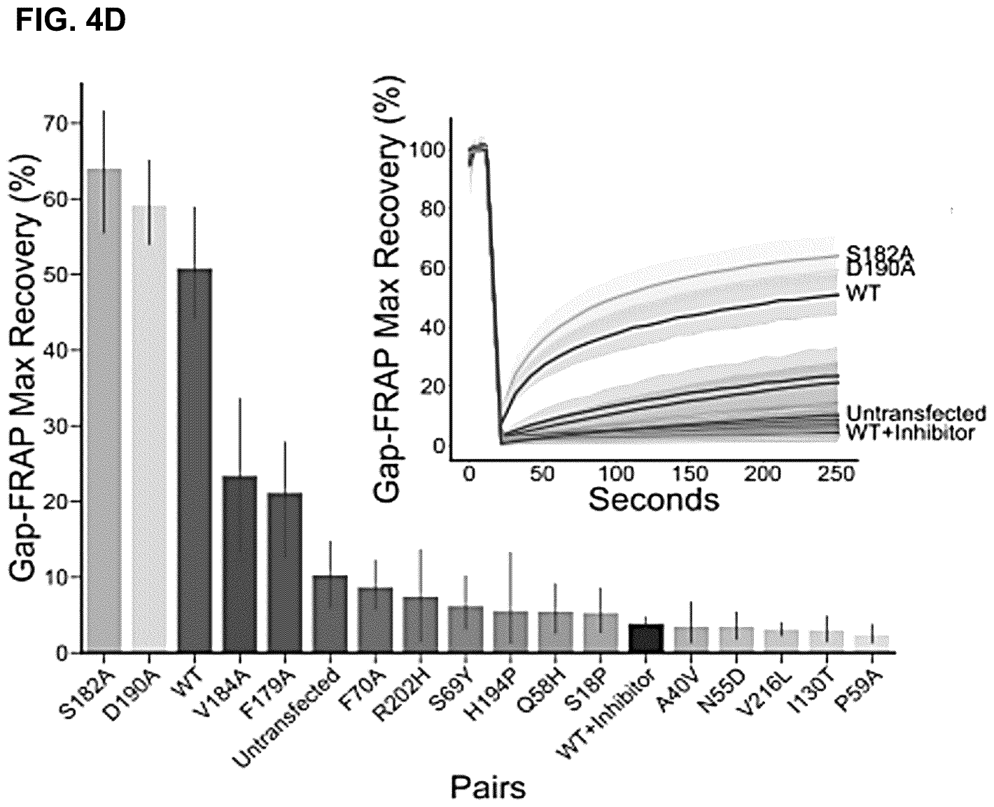

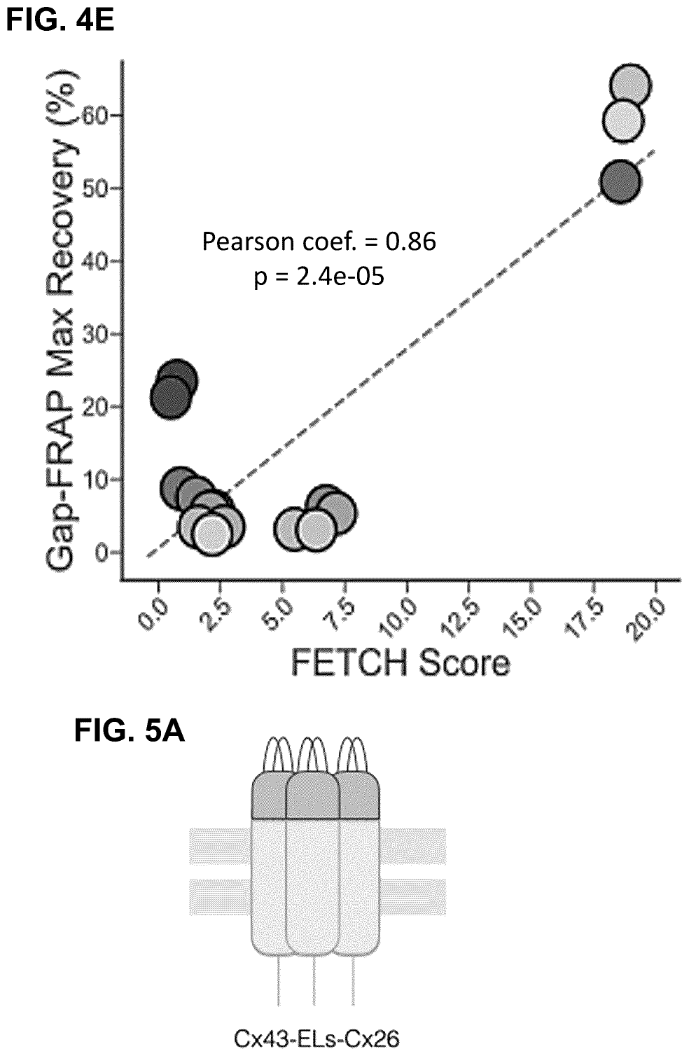

To further expand upon the capabilities of the FETCH method in identifying connexin docking interactions, 10 single amino acid mutations of Cx43 that are associated with the syndromic disease were selected to evaluate for homotypic and heterotypic (against WT-Cx43) docking. Of the 10 selected mutations associated with ODDD, 2 were localized to the intracellular N-terminus or loop (S18P and I130T), 2 were localized to transmembrane helices (A40V and V216L), 4 were localized to extracellular loop 1 (N55D, Q58H, P59A and S69Y), and 2 were localized to extracellular loop 2(H194P and R202H)(FIG. 4A). All 10 mutations were autosomal dominant and represented a range of observed channel perturbations when expressed in cultured cells and/or in mouse models. For example, the I130T and R202H mutations have been documented to form channels with reduced conduction (Lai, A, et al. (2006) J Cell Sci. 119:532-541; Mclachlan E, et al. (2005) Cell Commun Adhes. 12:279-292), whereas S18P, A40V, P59A and V216L mutations have been observed to result in a total loss of channel function (Laird D W (2014) Febs Letters. 588:1339-1348).

Because the FETCH score reflects hemichannel docking (and thus all the ‘upstream’ processes underlying gap junction formation (i.e., Cx expression levels, trafficking, stabilization and turnover rate)), the disease associated Cx43 mutations were expected to exhibit diminished FETCH scores as compared to the WT-Cx43 isoform. After generating a positive control distribution for homotypic Cx43 by co-plating Cx43-FP constructs in conditions of increasing DNA transfection quantities and increasing co-plated densities (n=33), the mutant proteins in homotypic FETCH experiments were evaluated. Upon analysis, Cx43 mutant proteins demonstrated lower homotypic FETCH scores than WT-Cx43 (FIG. 4B).

Since connexin hemichannel docking is a prerequisite for gap junction-mediated exchange of small molecules and ions between cells, it was reasoned that mutations that decreased the FETCH scores would also diminish intercellular permeability. A standard method of evaluating gap junction dependent intercellular communication is fluorescence recovery after photo-bleaching (FRAP), which relies on the permeability of Cx43 gap junctions to a tracer dye (calcein-AM) as an indicator of functional channels (Wade M H, et al. (1986) Science. 232:525-528; Martinez A D, et al. (2002) Circ Res. 90:1100-1107; Abbaci M, et al. (2007) Biotechnol J. 2:50-61) (FIG. 4C). Whether functional channels were being formed by mutant proteins, despite FETCH scores indicating diminished docking, was evaluated using FRAP.

Unsurprisingly, all selected Cx43 ODDD-associated mutant proteins showed significantly impaired dye transfer in FRAP experiments (FIG. 4D). Thus, negative FETCH scores correlated with poor gap junction functionality as determined by FRAP. Because an additional interest rested in identifying a correlation between positive FETCH scores and FRAP-determined functionality of mutant proteins, random alanine scanning mutations of the Cx43 ELs were made at positions not explicitly characterized as disease associated. 5 constructs were generated—F70A, F179A, S182A, V184A and D190A (FIG. 4A). Upon FETCH evaluation, two mutations that retained their ability to homotypically dock—S182A and D190A—were identified. Follow-up FRAP analysis demonstrated that random Cx43 mutants with positive homotypic FETCH scores were able to mediate dye transfer in FRAP assays, comparable to Cx43-WT (FIG. 4D). Finally, it was shown that FETCH scores correlated with FRAP-determined functionality (FIG. 4E). Combined, these results showed that FETCH can be used to detect dysfunctional gap junction intercellular communication and can characterize connexin mutant proteins for gap junction docking competency—a potential component to functional perturbation in pathological phenotypes.

### Example 4

### FETCH Evaluated Engineered Connexins for New Docking Features

Though full interrogation of the existing, expansive Cx interaction space remains of major value, a provocative application of the FETCH method is towards the development of engineered connexins with novel features. Since FETCH evaluates docking interactions, specificity of which is determined in the Cx extracellular loops (ELs), EL chimeras that would confer new docking features to an individual Cx were developed. Cx43 and Cx26 are two connexin isoforms shown to have positive homotypic FETCH scores (indicative of docking) while having a very low heterotypic FETCH score (FIG. 3)—making these two constructs amenable for engineering and evaluation.

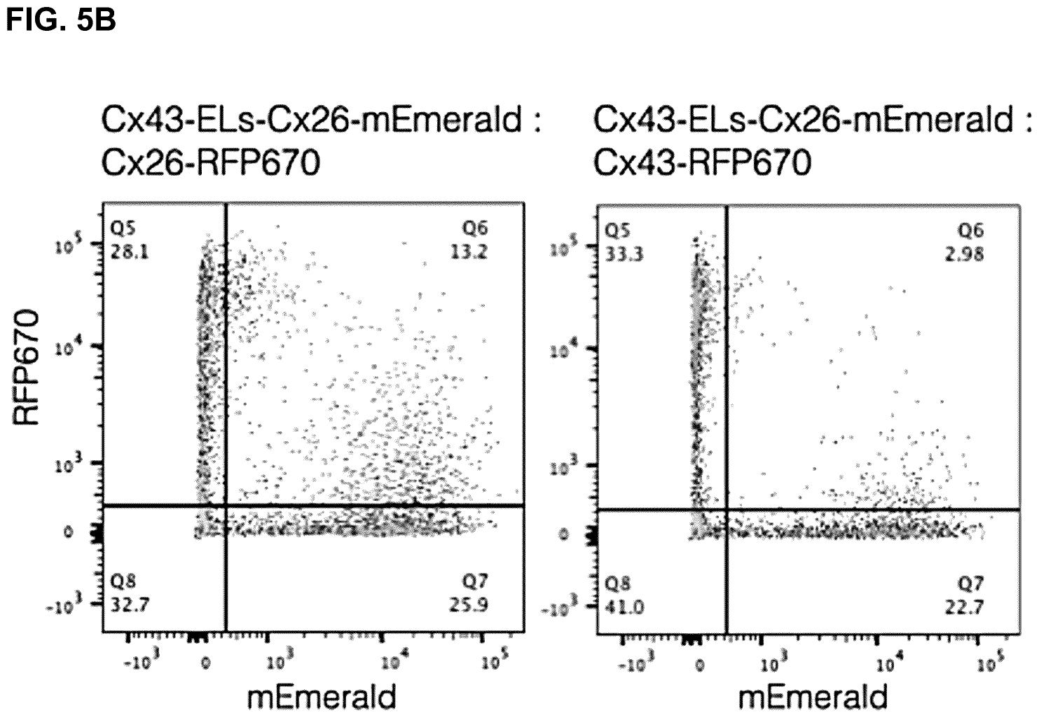

Accordingly, a chimeric construct consisting of the intracellular and transmembrane regions of Cx43 and the ELs of Cx26 was developed. (FIG. 5A). Such a connexin would be unique in that it would likely retain the trafficking, regulation, and conductivity features of Cx43, while acquiring the docking features of Cx26. Predictably, FETCH analysis demonstrated that the Cx43-ELs-Cx26 chimera preferentially docked with Cx26 and not Cx43 (FIG. 5B).

### Limitations of Experiments

For FETCH to be successful, cells must overexpress Cx-FP constructs to a sufficient degree to promote observable fluorescence exchange. Though HEK 293FT cells are rarely reported as vehicles for connexin studies, 293FT cells expressed significantly greater levels of exogenously introduced Cx-FP constructs than did 293 cells and HeLa cells. Moreover, though Cx43 gap junction plaques were identified in all the transfected cell lines, substantially larger surface area of plaques were observed in 293FTs, which can be due to the comparatively elevated protein expression (FIG. 7A-FIG. 7B). Critically, 293 and HeLa cell lines showed substantially lower FETCH scores, when expressing the otherwise robustly positive Cx43-FP constructs, thus, these cells lines are not recommended for FETCH analysis.

### Summary of Experiments

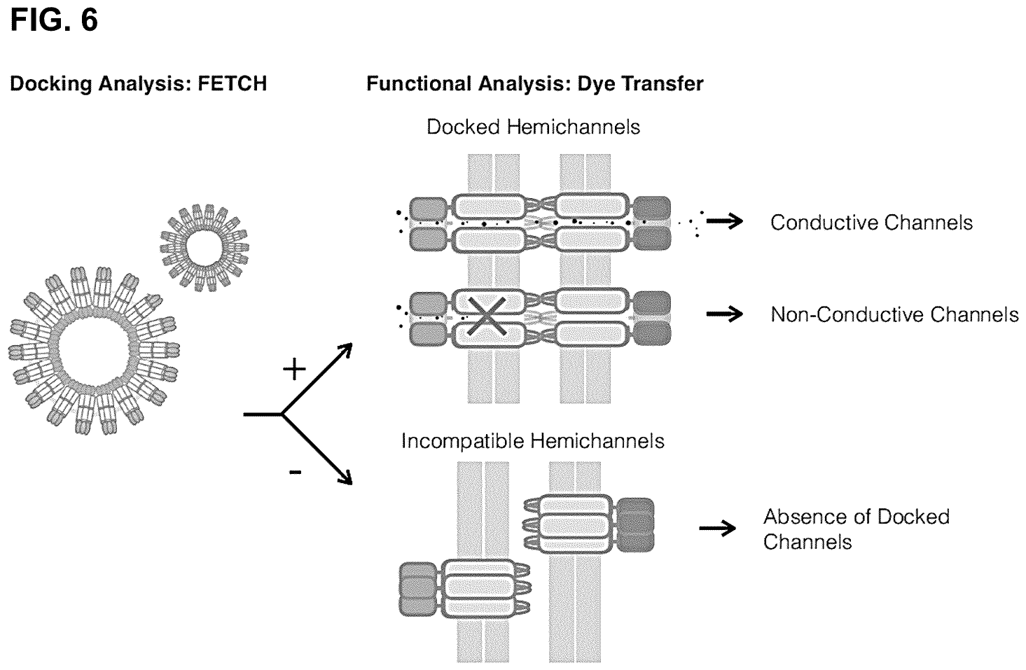

Disclosed here is FETCH, a novel method that employs flow cytometry to evaluate a natural product of gap junction turnover (connexosomes) as indicators of gap junction formation, a prerequisite for gap junction intercellular communication. While other methods primarily quantify gap junction conductive function in individual cell pairs or individual cells within a cell network, FETCH specifically captures hemichannel docking across a population of cells. By specifically evaluating hemichannel docking compatibility, FETCH can differentiate between two potential sources of gap junction non-functionality: (i) channels that are innately non-conductive and (ii) channels that fail to form, by precisely identifying the latter (FIG. 6). This distinction is critical for the dissection of complex mechanisms underlying both normal connexin physiology and connexin-associated pathologies.

To date, most studies have evaluated Cx43 (and other Cx isoform) mutations at the level of alterations in protein expression level, localization and conduction between cells. However, sufficient expression and plasma membrane localization do not guarantee docking. While the data provided herein did not identify Cx43 mutant proteins that successfully dock but are non-conductive, it stands to reason that mutations precisely characterized to have altered or disrupted gating properties would likely fit the scenario in which FETCH demonstrates docking and FRAP demonstrates non-conductivity.

While complementary to existing methods, FETCH makes advancements in terms of ease of use, accessibility, and time scale. Specifically, FETCH exploits the robust expression profile of the commercially available HEK 293FT cell line. Also, C-terminally tagged fluorescent vectors are widely-used and commercially available materials, and transient transfections avoid the costly and time-consuming generation and maintenance of stable cell lines. Also, FETCH is directly scalable for high-throughput evaluations as flow cytometry analysis can be completed from single sample tubes or multi-well plates.

The experiments provided herein show that FETCH can evaluate gap junction formation of numerous connexin proteins. Analysis of the entire complement of human connexin isoforms revealed that most isoforms generate positive FETCH scores when evaluated for homotypic docking. Furthermore, when evaluating heterotypic pairings of selected isoforms representing different isoform classes, positive FETCH scores were observed, which agrees with known heterotypic pairings such as Cx26/Cx32 and Cx43/Cx45, which generate heterotypic gap junctions in rodent liver and cardiomyocytes, respectively (Paul DL. (1986) J Cell Biol. 103(1):123-134; Veenstra R D, et al. (1986) Science. 233:972-974). Additionally, a potentially novel docking interaction when pairing Cx26/Cx36, was identified.

Finally, it was demonstrated that FETCH has utility in evaluating the docking ability of disease-relevant connexin mutant proteins. Specifically, several ODDD-associated Cx43 mutations were identified, and these Cx43 mutations result in docking perturbations that were also reflected in poor fluorescence recovery in GAP-FRAP functional analyses, indicating non-functionality.

Overall, these results demonstrate that FETCH is a valuable method that reliably evaluates connexin hemichannel docking interactions and can explore previously uncharacterized docking combinations of endogenous connexin isoforms, as well as evaluate docking features of mutant connexin proteins present in disease states.

Importantly, FETCH does not quantify hemichannel binding affinity. Instead, FETCH captures docking interactions in the context of several isoform specific variables (e.g., expression level, turnover rate, and plaque size) that likely contribute greatly to positive score variability amongst different isoforms. Thus, FETCH scores above a defined threshold are considered an indication of docking, but variation in the scores between different isoforms are not comparable. Furthermore, FETCH scores of individual isoforms can be increased by construct and condition optimization. On the other hand, discrepancies in FETCH score between wild-type and mutant counterparts of the same connexin isoform are comparable and directly identify perturbations in docking.

An important caveat is that FETCH conclusively identified docked, compatible connexin protein interactions, however, negative FETCH scores can reflect either non-compatible counterparts or a failure of one or both proteins to localize or fold properly.

Finally, FETCH does not reveal mechanism of pathology of evaluated disease-associated mutant proteins but instead sheds light on a single contributing aspect-docking ability of the mutant isoform. Thus, in an aspect, FETCH can be best utilized in conjunction with other biochemical and imaging analyses for comprehensive understanding of connexin behavior.

TABLE 5
Listing of Protein Sequences Provided in Sequence Listing.

| SEQ ID NO | Protein | UniProtKB No. |
|---|---|---|
| 1 | Gap Junction Protein Alpha 1 | P17302 |
| 2 | Gap Junction Protein Alpha 3 | Q9Y6H8 |
| 3 | Gap Junction Protein Alpha 4 | P35212 |
| 4 | Gap Junction Protein Alpha 5 | P36382 |
| 5 | Gap Junction Protein Alpha 8 | P48165 |
| 6 | Gap Junction Protein Alpha 9 | P57773 |
| 7 | Gap Junction Protein Alpha 10 | Q969M2 |
| 8 | Gap Junction Protein Beta 1 | P08034 |
| 9 | Gap Junction Protein Beta 2 | P29033 |
| 10 | Gap Junction Protein Beta 3 | O75712 |
| 11 | Gap Junction Protein Beta 4 | Q9NTQ9 |
| 12 | Gap Junction Protein Beta 5 | O95377 |
| 13 | Gap Junction Protein Beta 6 | O95452 |
| 14 | Gap Junction Protein Beta 7 | Q6PEYO |
| 15 | Gap Junction Protein Gamma 1 | P36383 |
| 16 | Gap Junction Protein Gamma 2 | Q5T442 |
| 17 | Gap Junction Protein Gamma 3 | Q8NFK1 |
| 18 | Gap Junction Protein Delta 2 | Q9UKL4 |
| 19 | Gap Junction Protein Delta 3 | Q8N144 |
| 20 | Gap Junction Protein Delta 4 | Q96KN9 |
| 21 | Gap Junction Protein Epsilon 1 | A6NN92 |

TABLE 6
Listing of Gene Sequences Provided in Sequence Listing.

| SEQ ID NO | Gene | NCBI Reference Sequence: Location |
|---|---|---|
| 22 | GJA1 | NG_008308.1: 5048-19129 |
| 23 | GJA3 | NG_016399.1: 4993-27790 |
| 24 | GJA4 | NC_000001.11: 34792999-34795747 |
| 25 | GJA5 | NG_009369.2: 5013-22176 |
| 26 | GJA8 | NG_016242.1: 4976-11451 |
| 27 | GJA9 | NC_000001.11: c38881587-38874069 |
| 28 | GJA10 | NC_000006.12: 89894469-89896120 |
| 29 | GJB1 | NG_008357.1: 5001-15305 |
| 30 | GJB2 | NG_008358.1: 5038-10506 |
| 31 | GJB3 | NG_008309.1: 5026-10176 |
| 32 | GJB4 | NG_016243.1: 5000-7587 |
| 33 | GJB5 | NC_000001.11: 34755047-34758512 |
| 34 | GJB6 | NG_008323.1: 5077-15434 |
| 35 | GJB7 | NC_000006.12: c87329278-87282980 |
| 36 | GJC1 | NC_000017.11: c44831364-44794104 |
| 37 | GJC2 | NG_011838.1: 5079-14975 |
| 38 | GJC3 | NG_016852.2: 5001-11355 |
| 39 | GJD2 | NC_000015.10: c34754998-34751032 |
| 40 | GJD3 | NC_000017.11: c40364737-40360652 |
| 41 | JD4 | NC_000010.11: 35605341-35608935 |
| 42 | GJE1 | NC_000006.12, 142132925-142135358 |

What is claimed is:

1. An isolated nucleic acid molecule, comprising: a nucleic acid sequence encoding a connexin protein and a nucleic acid sequence encoding a carboxy-terminal fluorescent label.

2. The isolated nucleic acid molecule of claim 1, wherein the encoded connexin protein comprises the sequence set forth in any one of SEQ ID NO:01-SEQ ID NO:21, or a fragment thereof.

3. The isolated nucleic acid molecule of claim 1, wherein the nucleic acid sequence can comprise the sequence set forth in any one of SEQ ID NO:22-SEQ ID NO:42, or a fragment thereof.

4. The isolated nucleic acid molecule of claim 1, wherein the nucleic acid sequence encoding a connexin protein is codon-optimized for expression in a human cell.

5. A method of evaluating gap junction formation, the method comprising: performing flow enabled tracking of connexosomes in HEK 293T cells (FETCH).

6. The method of claim 5, wherein performing FETCH comprises (i) generating fluorescent connexosomes, and (ii) using flow cytometry to track the fluorescent connexosomes in the one or more cell samples.

7. The method of claim 6, wherein generating one or more fluorescent connexosomes comprises transfecting a first population of cells with a first fluorescently-labeled, C-terminally fused connexin construct; and transfecting a second population of cells with a second fluorescently-labeled, C-terminally fused connexin construct.

8. The method of claim 7, wherein the first fluorescently-labeled, C-terminally fused connexin construct and the second fluorescently-labeled, C-terminally fused connexin comprise the isolated nucleic acid molecule of any one of claims 1-4.

9. The method of claim 7, further comprising incubating the transfected first and second populations of cells.

10. The method of claim 9, further comprising trypsinizing the incubated and transfected first and second populations of cells.

11. The method of claim 10, further comprising incubating the trypsinized first population of cells with the trypsinized second population of cells.

12. The method of claim 11, wherein incubating the combined populations of cells continues to hyperdensity and/or over-confluency.

13. The method of claim 12, further comprising trypsinizing the incubated combined population of cells, resuspending the trypsinized cells, and fixing the resuspended cells.

14. The method of claim 13, wherein, during flow cytometry, the cells are analyzed in two selection gates prior to fluorescence evaluation.

15. The method of claim 14, wherein analyzing the cells in two selection gates comprises (i) identifying cells by evaluating sample forward vs. side scatter area; and (ii) identifying single cells by evaluating cells that maintained a linear correlation of forward scatter height to forward scatter area.

16. The method of claim 14, wherein the flow cytometry generates a fluorescent profile for each cell sample.

17. The method of claim 16, wherein the fluorescence profile comprises 4 quadrants, wherein the 4 quadrants comprise Q1, Q2, Q3, and Q4.

18. The method of claim 17, further comprising establishing a FETCH score.

19. The method of claim 18, wherein the FETCH score comprises the proportion of dual colored-cells compared to fluorescent cells.

20. The method of claim 19, wherein the proportion comprises Q2/(Q1+Q2+Q3).

21. The method of claim 20, further comprising quantifying the fluorescence exchange between cells mediated by connexosomes.

22. The method of claim 20, further comprising confirming the relationship between the fluorescence exchange phenotype and the connexosome formation.

23. The method of claim 22, wherein the confirming the relationship between the fluorescence exchange phenotype and connexosome formation comprises using fluorescence-activated cell sorting (FACS) to collect cells from Q2 for microscopy analysis.

24. The method of claim 23, wherein FACS confirms the formation of dual-labeled connexosomes.

25. The method of claim 18, wherein the method identifies homotypic docking of hemichannels and/or heterotypic docking of hemichannels.

26. The method of claim 11, further comprising performing whole-cell, dual-patch clamp analysis on the combined populations of cells.

27. The method of claim 18, wherein the FETCH score reflects variation in (i) the expression level of the connexin constructs, (ii) the trafficking of the connexin constructs, (iii) the stability of the connexin constructs, and/or (iv) the turnover rate of the connexin constructs.
